(19) United States
(12) Patent Application Publication
(10) Pub. No.: US 2014/0342431 A1
(43) Pub. Date: Nov. 20, 2014

(54) Variant Alpha-Amylases and Methods of Use, Thereof

(71) Applicant: Danisco US Inc., Palo Alto, CA (US)

(72) Inventors: Luis Gustavo Cascao-Pereira, Redwood City, CA (US); Marc Kolkman, Oegsteest (NL)

(73) Assignee: Danisco US INC., Palo Alto, CA (US)

(21) Appl. No.: 14/364,408

(22) PCT Filed: Dec. 18, 2012
(86) PCT No.: PCT/US2012/070334

§ 371 Date: Jun. 11, 2014

Related U.S. Application Data
(60) Provisional application No. 61/579,356, filed on Dec. 22, 2011.

(51) Int. Cl. C11D 3/386; C12N 9/28

(52) U.S. Cl. CPC C11D 3/38618; C12N 9/2417
USPC 435/202; 435/264; 435/263; 536/23.2; 435/320.1; 435/252.3; 435/252.31; 435/252.35; 435/252.33; 435/254.2; 435/254.23; 435/254.21; 435/254.3; 435/254.7; 435/254.11; 435/254.8; 510/392

(57) ABSTRACT

Compositions and methods relating to variant alpha-amylases are described.

### PRIORITY

This application claims priority to U.S. Provisional Application Ser. No. 61/579,356, filed on Dec. 22, 2011, which is hereby incorporated by reference in its entirety.

### TECHNICAL FIELD

Disclosed are compositions and methods relating to variant α-amylase enzymes.

### BACKGROUND

Starch consists of a mixture of amylose (15-30% w/w) and amylopectin (70-85% w/w). Amylose consists of linear chains of α-1,4-linked glucose units having a molecular weight (MW) from about 60,000 to about 800,000. Amylopectin is a branched polymer containing α-1,6 branch points every 24-30 glucose units; its MW may be as high as 100 million.

Sugars from starch, in the form of concentrated dextrose syrups, are currently produced by an enzyme catalyzed process involving: (1) liquefaction (or viscosity reduction) of solid starch with an α-amylase into dextrins having an average degree of polymerization of about 7-10, and (2) saccharification of the resulting liquefied starch (i.e. starch hydrolysate) with amyloglucosidase (also called glucoamylase or GA). The resulting syrup has a high glucose content. Much of the glucose syrup that is commercially produced is subsequently enzymatically isomerized to a dextrose/fructose mixture known as isosyrup.

α-amylases hydrolyze starch, glycogen, and related polysaccharides by cleaving internal α-1,4-glucosidic bonds at random. α-amylases, particularly from Bacilli, have been used for a variety of different purposes, including starch liquefaction, textile desizing, starch modification in the paper and pulp industry, for baking and brewing, production of syrups for the food industry and in animal feed to increase digestability. These enzymes can also be used to remove starchy soils and stains during dishwashing and laundry washing.

One characterized α-amylase is that of alkaliphilic Bacillus sp. strain TS-23, which produces at least five kinds of enzymes exhibiting starch hydrolyzing activity (Lin et al., (1998) Biotechnol. Appl. Biochem. 28:61-68). The α-amylase of Bacillus sp. strain TS-23 has a pH optimum of 9 although it is stable over a broad pH range (i.e., pH 4.7 to 10.8). Its temperature optimum is 45° C., although the enzyme has activity at lower temperatures, e.g., 15-20° C. Variants of this enzyme have been described, e.g., in international patent applications WO 2009/061380, WO 2009/100102, and WO 2010/115028.

### SUMMARY

The present compositions and methods relate to variant α-amylase polypeptides, and methods of use, thereof.

In one aspect, a variant α-amylase polypeptide is provided, comprising at least one combinable mutation at a productive amino acid position; wherein: (i) the combinable mutation is a mutation that improves at least one desirable property of the variant α-amylase compared to the parental α-amylase, while not significantly decreasing either expression, activity, or stability of the variant α-amylase, compared to the parental α-amylase, (ii) the productive position is an amino acid position that can be substituted with a plurality of different amino acid residues, all of which substitutions result in a variant α-amylase that meets the requirements of (i), and (iii) the combinable mutation is listed in Table C or Table D, which uses SEQ ID NO: 2 for numbering.

In some embodiments, the variant combinable mutation has a performance property listed in Table A. In some embodiments, the combinable mutation produces a variant wherein the minimum performance indices (PI) relative to the parental amylase for (i) protein expression, (ii) activity, (iii) microswatch activity, and (iv) detergent stability or thermostability are greater than or equal to 0.9, and in addition the PI for any one of these properties is greater than or equal to 1.0. In some embodiments, the combinable mutation produces a variant wherein the minimum performance indices (PI) relative to the parental amylase for (i) protein expression, (ii) activity, (iii) microswatch activity, and (iv) detergent stability or thermostability are greater than or equal to 0.8, and in in addition have a PI for any one of these tests that is greater than or equal to 1.2. in some embodiments, the combinable mutation produces a variant wherein the minimum performance indices (PI) relative to the parental amylase for (i) protein expression, (ii) activity, (iii) microswatch activity, and (iv) detergent stability or thermostability are greater than or equal to 0.5, and in in addition have a PI for any one of these tests that is greater than or equal to 1.5.

In some embodiments, the combinable mutation has a sustainability score of +++, ++++, or +++++. In some embodiments, the combinable mutation has a sustainability score of ++++, or +++++. In some embodiments, the combinable mutation has a sustainability score of +++++. In some embodiments, the combinable mutation has a productivity score of 1 or 2.

In some embodiments, the parental α-amylase has at least 60% amino acid sequence identity to the amino acid sequence of SEQ ID NO: 1 or SEQ ID NO: 2. In some embodiments, the parental α-amylase has at least 70% amino acid sequence identity to the amino acid sequence of SEQ ID NO: 1 or SEQ ID NO: 2. In some embodiments, the parental α-amylase has at least 80% amino acid sequence identity to the amino acid sequence of SEQ ID NO: 1 or SEQ ID NO: 2. In some embodiments, the parental α-amylase has at least 90% amino acid sequence identity to the amino acid sequence of SEQ ID NO: 1 or SEQ ID NO: 2.

In another aspect, a composition comprising any of the foregoing variant amylases is provided. In some embodiments, the composition is effective for removing starchy stains from laundry, dishes, or textiles. In some embodiments, the composition comprises a surfactant. In some embodiments, the composition is a detergent composition. In some embodiments, the composition is a laundry detergent or a laundry detergent additive. In some embodiments, the composition is manual or automatic dishwashing detergent.

In another aspect, a method for removing a starchy stain or soil from a surface is provided, comprising: incubating the surface in the presence of a aqueous composition comprising an effective amount of the variant amylase of any of the claims 1-13, allowing the polypeptide to hydrolyze starch components present in the starchy stain to produce smaller starch-derived molecules that dissolve in the aqueous composition, and rinsing the surface, thereby removing the starchy stain from the surface.

In some embodiments, the aqueous composition further comprises a surfactant. In some embodiments, the surface is a textile surface. In some embodiments, the surface is on dishes. In some embodiments, the surface is a soiled hard surface.

In another aspect, an isolated polynucleotide encoding any of the forementioned polypeptides is provided, as is an expression vector comprising the polynucleotide and a host cell comprising the expression vector.

These and other aspects and embodiments of the compositions and methods will be apparent from the present description and drawings.

### BRIEF DESCRIPTION OF THE DRAWINGS

FIG. 1 is a map of plasmid pHPLT-Amy TS23t.

### BRIEF DESCRIPTION OF THE SEQUENCES

SEQ ID NO: 1 sets forth the amino acid sequence of the mature form of Bacillus sp. strain TS-23amylase.

SEQ ID NO: 2 sets forth the amino acid sequence of the mature form of a C-terminal truncated form of Bacillus sp. strain TS-23amylase.

### DETAILED DESCRIPTION

Described are compositions and methods relating to variant α-amylase enzymes. The variant amylases include mutations to impart a performance benefit, for example, increased hydrolysis of a starch substrate, increased cleaning performance, increased thermal stability, increased storage stability, increased solubility, an altered pH profile, decreased calcium dependence, and/or increased expression. In some eases, the performance benefit is realized at low temperatures.

The subject α-amylases are variants of Bacillus sp. strain TS-23 amylase (i.e., AmyTS23), or variants of amylases that share at least 60%, at least 70%, at least 80%, or even at least 90%, sequence identity with AmyTS23.

Exemplary applications for the present amylases are in the preparation of cleaning compositions, such as detergent compositions for cleaning laundry, dishes [including manual and automatic dishwashing (ADW)], and other surfaces, for textile processing (e.g., desizing), in animal feed for improved digestibility, and for starch liquefaction and saccharification. These and other aspects of the compositions and methods are described in detail, below.

### 1. DEFINITIONS AND ACRONYMS

In accordance with this detailed description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

The present document is organized into a number of sections for ease of reading; however, the reader will appreciate that statements made in one section may apply to other sections. In this manner, the headings used for different sections of the disclosure should not be construed as limiting.

Unless defined otherwise, all technical and scientific terms used herein have the same meaning as commonly understood by one of ordinary skill in the art. The following abbreviations and/or terms are defined for clarity:

### 1.1 Abbreviations/Acronyms

The following abbreviations/acronyms have the following meanings unless otherwise specified:

AE alcohol ethoxylate

AEO alcohol ethoxylate

AEOS alcohol ethoxysulfate

AES alcohol ethoxysulfate

AOS α-olefinsulfonate

AS alkyl sulfate

cDNA complementary DNA

CMC carboxymethylcellulose

DNA deoxyribonucleic acid

DTMPA diethylenetriaminepentaacetic acid

EC enzyme commission

EDTA ethylenediaminetetraacetic acid

EO ethylene oxide (polymer fragment)

GA glucoamylase

IPTG isopropyl β-D-thiogalactoside

kDa kiloDalton

LAS linear alkylbenzenesulfonate

LAT B. licheniformis amylase

MW molecular weight

MWU modified Wohlgemuth unit; 1.6×10⁻⁵ g/MWU=unit of activity

NOBS nonanoyloxybenzenesulfonate

NTA nitriloacetic acid

OxAm Purastar HPAM 5000L (Danisco US Inc.)

PEG polyethyleneglycol

pI isoelectric point

PVA poly(vinyl alcohol)

PVP poly(vinylpyrrolidone)

RNA ribonucleic acid

SAS alkanesulfonate

SDS-PAGE sodium dodecyl sulfate polyacrylamide gel electrophoresis

sp. species

TAED tetraacetylethylenediamine

w/v weight/volume

w/w weight/weight

v/v volume/volume

wt % weight percent

° C. degrees Centigrade

H₂O water

dH₂O or DI deionized water

dIH₂O deionized water, Milli-Q filtration

g or gm grams

μg micrograms

mg milligrams

kg kilograms

μL and μl microliters

mL and ml milliliters

mm millimeters

μm micrometer

M molar

mM millimolar

μM micromolar

U units

sec seconds

min(s) minute/minutes

hr(s) hour/hours

DO dissolved oxygen

Ncm Newton centimeter

ETOH ethanol

eq. equivalents

N normal

ds or DS dry solids content

### 1.2 Definitions

The terms “amylase” or “amylolytic enzyme” refer to an enzyme that is, among other things, capable of catalyzing the degradation of starch. α-amylases are hydrolases that cleave the α-D-(1→4) β-glycosidic linkages in starch. Generally, α-amylases (EC 3.2.1.1; α-D-(1→4)-glucan glucanohydrolase) are defined as endo-acting enzymes cleaving α-D-(1→4) β-glycosidic linkages within the starch molecule in a random fashion yielding polysaccharides containing three or more (1-4)-α-linked D-glucose units. In contrast, the exo-acting amylolytic enzymes, such as β-amylases (EC 12.1.2; α-D-(1→4)-glucan maltohydrolme) and some product-specific amylases like maltogenic α-amylase (EC 3.2.1.133) cleave the polysaccharide molecule from the non-reducing end of the substrate. β-amylases, α-glucosidases (EC 3.2.1.20; α-D-glucoside glucohydrolase), glucoamylase (EC 3.2.1.3; α-D-(1→4)-glucan glucohydrolase), and product-specific amylases like the maltotetraosidases (EC 3.2.1.60) and the maltohexaosidases (EC 3.2.1.98) can produce malto-oligosaccharides of a specific length. Some bacterial α-amylases predominantly produce maltotetraose (G4), maltopentaose (G5) or maltohexaose (G6) from starch and related α-1,4-glucans, while most α-amylases further convert them to glucose and or maltiose as final products.

As used herein the term “starch” refers to any material comprised of the complex polysaccharide carbohydrates of plants, comprised of amylose and amylopectin with the formula (C₆H₁₀O₅)ₓ, wherein X can be any number. The term includes plant-based materials such as grains, grasses, tubers and roots, and more specifically materials obtained from wheat, barley, corn, rye, rice, sorghum, brans, cassava, millet, potato, sweet potato, and tapioca.

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

The term “variant,” with respect to a polypeptide, refers to a polypeptide that differs from a specified wild-type, parental, or reference polypeptide in that it includes a man-made substitution, insertion, or deletion at one or more amino acid positions. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context.

The term “recombinant,” when used in reference to a subject cell, nucleic acid, protein or vector, indicates that the subject has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell, or express native genes at different levels or under different conditions than found in nature.

As used herein, “combinable mutations” are mutations at any amino acid position that can be used to make combinatorial variants. Combinable mutations improve at least one desired property of the molecule (in this case, an amylase), while not significantly decreasing either expression, activity, or stability. Combinable mutations can be grouped as follows:

### Group A:

A mutation that produces a variant wherein the minimum performance indices (PI) relative to a defined parental protein for: (i) protein expression, (ii) activity, (iii) CS-28 microswatch activity at pH 8 (16° C., 32° C., or 50° C.) or pH10 (16° C. or 50° C.), and (iv) detergent stability or thermostability are greater than or equal to 0.9, and in addition have a PI for any one of these tests that is greater than or equal to 1.0.

### Group B:

A mutation that produces a variant wherein the minimum performance indices (PI) relative to a defined parental protein for: (i) protein expression, (ii) activity, (iii) CS-28 microswatch activity at pH 8 (16° C., 32° C., or 50° C.) or pH10 (16° C. or 50″C), and (iv) detergent stability or thermostability are greater than or equal to 0.8, and in in addition have a PI for any one of these tests that is greater than or equal to 1.2.

### Group C:

A mutation that produces a variant wherein the minimum performance indices (PI) relative to a defined parental protein for: (i) protein expression, (ii) activity, (iii) CS-28 microswatch activity at pH 8 (16° C., 32° C., or 50° C.) or pH10 (16° C. or 50° C.), and (iv) detergent stability or thermostability are greater than or equal to 0.5, and in addition have a PI for any one of these tests that is greater than or equal to 1.5.

The properties of combinable mutations are summarized in the following Table.

TABLE A
Performance properties for each group of combinable mutations

| | Performance Index (PI) | | | |
|---|---|---|---|---|
| | Express- | Cleaning | Stability (detergent | Minimum PI in one |
| Group | ion | pH 8 or 10) | or thermal) | or more tests |
| A | ≧0.9 | ≧0.9 | ≧0.9 | X ≧ 1.0 |
| B | ≧0.8 | ≧0.8 | ≧0.8 | X ≧ 1.2 |
| C | ≧0.5 | ≧0.5 | ≧0.5 | X ≧ 1.5 |

Preferred combinable mutations are at “productive positions,” as described, below. In the case of the present amylases, “activity” refers to amylase activity, which can be measured as described, herein.

As used herein, “productive positions” are amino acid positions that are tolerant to substitution with different amino acid residues, wherein the resulting variants meet a set of performance criteria for combinability, as set forth above. Productive positions can be assigned a Productivity Score as follows:
1, Positions where less than 15% of the substitutions at a given position fall within groups A, B, or C are given a Productivity Score of “1”.
2. Positions where less than 40%, but greater than, or equal to 15% of the substitutions at a given position fall within groups A, B, or C are given a Productivity Score of “2”.
3. Positions where less than 75%, but greater than, or equal to 40% of the substitutions at given position fall within groups A, B, or C are given a Productivity Score of “3”.
4. Positions where 75% or more of the substitutions at a given position fall within groups A, B, or C are given a Productivity Score of “4”.

Preferred productive positions are combinable mutations.

As used herein, “suitability score” refers to the ability of one or more combinable mutations to be used to make combinatorial variants, based on the performance criteria for combinability, (i.e., A, B, and C, as set forth, above) in which each of the mutations fall. A higher suitability score indicates a mutation or mutations that are more suitable for use in making combinatorial variants. Suitability scores are described in the following Table.

TABLE B
Definitions of suitability scores

| Substitutions Occur in | |
|---|---|
| Group(s) | Suitability Score |
| A, B and C | +++++ |
| A and B | ++++ |
| A or (B and C) | +++ |
| B | ++ |
| C | + |

The terms “recovered,” “isolated,” and “separated,” refer to a compound, protein (polypeptides), cell, nucleic acid, amino acid, or other specified material or component that is removed from at least one other material or component with which it is naturally associated as found in nature.

As used herein, the term “purified” refers to material (e.g., an isolated polypeptide or polynucleotide) that is in a relatively pure state, e.g., at least about 90% pure, at least about 95% pure, at least about 98% pure, or even at least about 99% pure.

The term “enhanced stability” or “increased stability” in the context of an oxidating environment, the presence of chelators, the presence of detergents, exposure to elevated temperatures, and/or exposure to pH extremes, means that a subject amylase retains more amylolytic activity over time compared to another (i.e., reference) amylase.

The terms “thermostable” and “thermostability,” with reference to an enzyme, refer to the ability of the enzyme to retain activity after exposure to an elevated temperature. The thermostability of an enzyme, such as an amylase enzyme, is measured by its half-life (t_{1/2}) given in minutes, hours, or days, during which half the enzyme activity is lost under defined conditions. The half-life may be calculated by measuring residual amylase activity following exposure to (i.e., challenge by) an elevated temperature.

As used herein, the expression “substantially 100% stability in the presence of a protease-containing commercial laundry detergent composition,” or similar language, means at least 95%, at least 96%, at least 97%, at least 98%, at least 99%, and up to 100% stability. Each and any of these values may be specified with reference to this stability property.

A “pH range,” with reference to an enzyme, refers to the range of pH values under which the enzyme exhibits catalytic activity.

As used herein, the terms “pH stable” and “pH stability,” with reference to an enzyme, relate to the ability of the enzyme to retain activity over a wide range of pH values for a predetermined period of time (e.g., 15 min., 30 min., 1 hour).

As used herein, the term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-ea/boxy terminal orientation (i.e., N→C).

The term “nucleic acid” encompasses DNA, RNA, heteroduplexes, and synthetic molecules capable of encoding a polypeptide. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

By “homologue” shall mean an entity having a specified degree of identity with the subject amino acid sequences and the subject nucleotide sequences. A homologous sequence is taken to include an amino acid sequence that is at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or even at least 99% identical to the subject sequence, using the well-known sequence alignment tool Clustal V with default parameters. Typically, homologues will include the same active site residues as the subject amino acid sequence, unless otherwise specified.

As used herein, “hybridization” refers to the process by which one strand of nucleic acid base pairs with a complementary strand, as occurs during blot hybridization techniques and PCR techniques. Stringent hybridization conditions are exemplified by the following: 65° C. and 0.1×SSC (where 1×SSC=0.15 M NaCl, 0.015 M Na₃ citrate, pH 7.0).

As used herein, a “synthetic” molecule is produced by in vitro chemical or enzymatic synthesis rather than by an organism.

As used herein, the terms “transformed,” “stably transformed,” and “transgenic,” used with reference to a cell means that the cell contains a non-native heterologous) nucleic acid sequence integrated into its genome or carried as an episome that is maintained through multiple generations.

The term “introduced” in the context of inserting a nucleic acid sequence into a cell, means “transfection”, “transformation” or “transduction,” as known in the art.

A “host strain” or “host cell” is an organism into which an expression vector, phage, virus, or other DNA construct, including a polynucleotide encoding a polypeptide of interest (e.g., an amylase) has been introduced. Exemplary host strains are bacterial cells. The term “host cell” includes protoplasts created from cells, such as those of a Bacillus sp.

The term “heterologous” with reference to a polynucleotide or protein refers to a polynucleotide or protein that does not naturally occur in a host cell.

The term “endogenous” with reference to a polynucleotide or protein refers to a polynucleotide or protein that occurs naturally in the host cell.

As used herein, the term “expression” refers to the process by which a polypeptide is produced based on a nucleic acid sequence. The process includes both transcription and translation.

A “selective marker” or “selectable marker” refers to a gene capable of being expressed in a host to facilitate selection of host cells carrying the gene. Examples of selectable markers include but are not limited to antimicrobials (e.g., hygromycin, bleomycin, or chloramphenicol) and/or genes that confer a metabolic advantage, such as a nutritional advantage on the host cell.

A “vector” refers to a polynucleotide sequence designed to introduce nucleic acids into one or more cell types, Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences that control termination of transcription and translation.

The term “operably linked” means that specified components are in a relationship (including but not limited to juxtaposition) permitting them to function in an intended manner. For example, a regulatory sequence is operably linked to a coding sequence if the expression of the coding sequence is under control of the regulatory sequences.

A “signal sequence” is a sequence of amino acids attached to the N-terminal portion of a protein, which facilitates the secretion of the protein outside the cell. The mature form of an extracellular protein lacks the signal sequence, which is cleaved off during the secretion process.

As used herein, “biologically active” refer to a sequence having a specified biological activity, such an enzymatic activity.

“Water hardness” is a measure of the minerals (e.g., calcium and magnesium) present in water.

As used herein, a “swatch” is a piece of material such as a fabric that has a stain applied thereto. The material can be, for example, fabrics made of cotton, polyester or mixtures of natural and synthetic fibers. The swatch can further be paper, such as filter paper or nitrocellulose, or a piece of a hard material such as ceramic, metal, or glass. For amylases, the stain is starch based, but can include blood, milk, ink, grass, tea, wine, spinach, gravy, chocolate, egg, cheese, clay, pigment, oil, or mixtures of these compounds.

As used herein, a “smaller swatch” is a section of the swatch that has been cut with a hole punch device, e.g., a custom manufactured 96-hole punch device, where the pattern of the multi-hole punch is matched to standard 96-well microtiter plates, or the section has been otherwise removed from the swatch. The swatch can be of textile, paper, metal, or other suitable material. The smaller swatch can have the stain affixed either before or after it is placed into the well of a 24-, 48- or 96-well microtiter plate. The smaller swatch can also be made by applying a stain to a small piece of material. For example, the smaller swatch can be a stained piece of fabric ⅝″ or 0.25″ in diameter. The custom manufactured punch is designed in such a manner that it delivers 96 swatches simultaneously to all wells of a 96-well plate. The device allows delivery of more than one swatch per well by simply loading the same 96-well plate multiple times. Multi-hole punch devices can be conceived of to deliver simultaneously swatches to any format plate, including but not limited to 24-well, 48-well, and 96-well plates. In another conceivable method, the soiled test platform can be a bead made of metal, plastic, glass, ceramic, or another suitable material that is coated with the soil substrate. The one or more coated beads are then placed into wells of 96-, 48-, or 24-well plates or larger formats, containing suitable buffer and enzyme.

As used herein, “a cultured cell material comprising an α-amylase polypeptide,” or similar language, refers to a cell lysate or supernatant (including media) that includes an α-amylase polypeptide as a component. The cell material is preferably from a heterologous host that is grown in culture for the purpose of producing the α-amylase polypeptide.

All references cited herein are expressly incorporated by reference.

### 2. AMYLASE POLYPEPTIDES AND NUCLEIC ACIDS

The mature form of the α-amylase from Bacillus sp. strain TS-23 (i.e., AmyTS23) has the amino acid sequence of SEQ ID NO: 1:

| | |
|---|---|
| NTAPINETMM QYFEWDLPND GTLWTKVKNE AANLSSLGIT ALWLPPAYKG | 50 |
| TSQSDVGYGV YDLYDLGEFN QKGTIRTKYG TKTQYIQAIQ AAKAAGMQVY | 100 |
| ADVVFNHKAG ADGTEFVDAV EVDPSNRNQE TSGTYQIQAW TKFDFPGRGN | 150 |
| TYSSFKWRWY HFDGTDWDES RKLNRIYKFR STGKAWDWEV DTENGNYDYL | 200 |
| MFADLDMDHP EVVTELKNWG TWYVNTTNID GFRLDAVKNI KYSFFPDWLT | 250 |
| YVRNQTGKNL FAVGEFWSYD VNKLHNYITK TNGSMSLFDA PLHNNFYTAS | 300 |
| KSSGYFDMRY LLNNTLMKDQ PSLAVTLVDN HDTQPGQSLQ SWVEPWFKPL | 350 |
| AYAFILTRQE GYPCVFYGDY YGIPKYNIPG LKSKIDPLLI ARRDYAYGTQ | 400 |
| RDYIDHQDII GWTREGIDTK PNSGLAALIT DGPGGSKWMY VGKKHAGKVF | 450 |
| YDLTGNRSDT VTINADGWGE FKVNGGSVSI WVAKTSNVTF TVNNATTTSG | 500 |
| QNVYVVANIP ELGNWNTANA IKMNPSSYPT WKATIALPQG KAIEFKFIKK | 550 |
| DQAGNVIWES TSNRTYTVPF SSTGSYTASW NVP | 583 |

An enzymatically active C-terminal-truncated for of the mature α-amylase from Bacillus sp. strain TS-23 (i.e., AmyTS23t or BASE) has the amino acid sequence of SEQ ID NO: 2:

NTAPINETMMQYFEWDLPNDGTLWTKVKNEAANLSSLGITALWLPPAYK
GTSQSDVGYGVYDLYDLGEFNQKGTIRTKYGTKTQYIQAIQAAKAAGMQ
VYADVVFNHKAGADGTEFVDAVEVDPSNRNQETSGTYQIQAWTKFDFPG
RGNTYSSFKWRWYHFDGTDWDESRKLNRIYKFRSTGKAWDWEVDTENGN
YDYLMFADLDMDHPEVVTELKNWGTWYVNTTNIDGFRLDAVKHIKYSFF
PDWLTYVRNQTGKNLFAVGEFWSYDVNKLHNYITKTNGSMSLFDAPLHN
NFYTASKSSGYFDMRYLLNNTLMKDQPSLAVTLVDNHDTQPGQSLQSWV
EPWFKPLAYAFILTRQEGYPCVFYGDYYGIPKYNIPGLKSKIDPLLIAR
RDYAYGTQRDYIDHQDIIGWTREGIDTKPNSGLAALITDGPGGSKWMYV
GKKHAGKVFYDLTGNRSDTVTINADGWGEFKVNGGSVSIWVAK

Variants of Bacillus sp. strain TS-23 have been described, e.g., in international patent applications WO 2009/061380, WO 2009/100102, and WO 2010/115028. The present compositions and method relate to further variants of Bacillus sp. strain TS-23, which satisfy the various criteria described, herein. Preferred mutations are combinable substitutions at productive positions, in which the amino acid residue present in the parental amylase (i.e., the “wild-type” residue) is replaced with a different amino acid residue. Specific substitutions are listed in Tables C and D.

TABLE C
Combinable substitutions at productive positions

| Amino | |
|---|---|
| acid | |
| Position | Substitutions |
| 12 | Y, D, H, Q, T, G, S |
| 13 | F, Y, S |
| 20 | D, N |
| 23 | L, A, E, F, G, M, N, S, T, W, Y, Q |
| 27 | V, E, G, N, S, T, Q |
| 31 | A, G, H, P, T, Y, Q, S |
| 33 | N, D, G, P |
| 34 | L, H, N, Q |
| 40 | T, R |
| 41 | A, D, Q, R, S |
| 43 | W, Y |
| 44 | L, T |
| 48 | Y, Q, T |
| 63 | L , V, E, M, S |
| 64 | Y, T, S |
| 66 | L, I |
| 68 | E, D, N |
| 74 | T, L, W |
| 79 | Y, H, W |
| 81 | T, H |
| 88 | A, S |
| 89 | I, M, V, A |
| 92 | A,G |
| 96 | G, A, C, E, K |
| 97 | M, F, V |
| 106 | N, D |
| 109 | A, E, T |
| 117 | V, E, Q, P, W, A, S |
| 120 | V, A, N |
| 122 | V, C |
| 133 | G, N, D, P, E, H |
| 139 | A, P |
| 146 | P, A |
| 173 | L, Q, W |
| 180 | R, I, M, V, A, D, K, N, Q, T, C, G, W |
| 181 | S, A, G, C, D, H, M, P, Q, T |
| 194 | N, F, H |
| 209 | H, E, Y, A, C, W, M, N, Q, R |
| 212 | V, P |
| 213 | V, T, P |
| 215 | E, H |
| 218 | N, S |
| 223 | Y, F, L, S |
| 224 | V, K, R, Q |
| 225 | N, D |
| 226 | T, A, H, N, Q |
| 229 | I, V, S |
| 232 | F, W |
| 242 | Y, F |
| 243 | S, D, Q, A |
| 244 | F, H, V, N, S, L, M, T |
| 245 | F, N, S, V, A, M, Q, T |
| 249 | L, Q |
| 251 | Y, H, M, Q, R, S, P |
| 252 | V, L |
| 253 | R, K, T, H |
| 256 | T, N, D, Q, R, Y |
| 258 | K, Q, C, N |
| 260 | L, M, R |
| 262 | A, G, M |
| 271 | V, Y, I, P, R, S, M, T, A, D, G, W, H, Q |
| 282 | N, D, L, M, E |
| 285 | M, Q, T, V, I, A |
| 286 | S, D, H, Q, T, N, R |
| 287 | L, I |
| 294 | N, R, E |
| 295 | N, R |
| 302 | S, E, K, Q, T, L, D |
| 304 | G, N, Q |
| 312 | L, I, K, M, Q, R, S, T |
| 313 | N, R, K, T |
| 321 | P, N, R, T, A |
| 326 | T, S |
| 327 | L, I, M |
| 331 | H, Q, C, G, T, V |
| 335 | P, D, G, H, I, L, M, N, Q, T, K |
| 341 | S, D, R, Q, E, H, N |
| 342 | W, Q, L, S |
| 345 | P, D |
| 346 | W, A, F |
| 349 | P, Q, T |
| 350 | L, I, M, S, Q |
| 353 | A, S |
| 354 | F, H, L, M, G, T |
| 355 | I, T |
| 357 | T, A |
| 358 | R, H |
| 360 | E, A, Q, S, F |
| 362 | Y, A, I, S, D, H, K, T, R, E |
| 364 | C, A, D, H, S, T, Q, M |
| 366 | F, D |
| 367 | Y, E, H, Q, F |
| 371 | Y, A, H, N, T, R |
| 378 | I, A, H, M, N, S |
| 380 | G, D, E, N, P, Q, R, K |
| 383 | S, I, R, T, L |
| 384 | K, Y |
| 385 | I, L, M |
| 386 | D, Q |
| 387 | P, A, R, T |
| 391 | A, S |
| 395 | Y, F, H, N, Q, R |
| 396 | A, G, S |
| 400 | Q, E, H, N |
| 404 | I, E, S |
| 405 | D, S |
| 406 | H, D |
| 410 | I, A |
| 413 | T, S, A |
| 415 | E, D |
| 417 | I, D, E, G, K, L, M, Q, S, T, V, Y |
| 418 | D, H, Q, S, A, G |
| 420 | K, F, H, L, N, Q, R, S, Y, A |
| 421 | P, A, C, D, E, F, G, H, I, K, L, N, T, W, Y |
| 422 | N, A, C, D, H, I, L, Q, R, S, T, W, Y |
| 423 | S, A, D |
| 424 | G, S |
| 425 | L, A, F, I, T, V, Y |
| 427 | A, G, T |
| 429 | I, E, T |
| 430 | T, D |
| 434 | G, H, I, K, N, T, V, A, Q |
| 435 | G, C, M, T |
| 440 | Y, C, E, F, H, L, P, Q, S, T, W, D, G, N, R |
| 442 | G, D |
| 443 | K, A, C, D, E, F, G, H, Q, R, S, T, W, Y |
| 449 | V, A, C, D, E, G, H, K, M, P, T, Y, \Q |
| 454 | T, A, Q |
| 455 | G, A, D, K, N, S |
| 456 | N, T, H |
| 457 | R, A, L, Q, T, W |
| 458 | S, A, D, F, K, Q, T, V, W, L |
| 460 | T, A, E, I, L, P, Q |
| 461 | V, A, H, Q, T |
| 462 | T, D, K, P, R, V |
| 463 | I, A, M, V, S |
| 464 | N, D, G, H, K, L, M, TA |
| 466 | D, E, Q, S, T |
| 468 | W, A, C, F, G, H, L, M, N, Q, R, T, Y |
| 469 | G, A, S |
| 471 | F, M, Y |
| 472 | K, A, I, R, T, D |
| 474 | N, A, D, E, F, I, K, M, S, L, T |
| 476 | G, A, N, P, Q, S, T, V, Y |
| 477 | S, H, K, R |
| 478 | V, C, L, M, W, N, Q, S, T, A, I |
| 479 | S, A, G, T, C |
| 482 | V, L, H |

TABLE D
Combinable substitutions at productive positions

| Amino | |
|---|---|
| Acid | |
| Position | Substitution(s) |
| 15 | H |
| 25 | G |
| 50 | D |
| 59 | C, K |
| 107 | C, S |
| 140 | K |
| 162 | M |
| 165 | F |
| 166 | C, G, H, P, Q |
| 177 | H |
| 179 | I |
| 203 | K, N, D, Q |
| 236 | E |
| 337 | P, V, Y |
| 339 | C, F, S, T |
| 484 | Q, S |

In some embodiments, the present variant amylases include one or more of the substitutions listed in Table C and/or Table D. In some embodiments, the present variant amylases include, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more of the substitutions listed in Table C and/or Table D.

In some embodiments, the present amylase is a variant of Bacillus sp. strain IS-23 amylase having a defined degree of amino acid sequence homology/identity to SEQ ID NO: 1, for example, at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or even at least 99% amino acid sequence homology/identity.

In some embodiments, the present amylase is a variant of Bacillus sp. strain TS-23 amylase having a C-terminal truncations, as exemplified by the amino acid sequence of SEQ ID NO: 2, and having a defined degree of amino acid sequence homology/identity to SEQ ID NO: 2, for example, at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or even at least 99% amino acid sequence homology/identity.

In addition to the mutations described, herein, the present amylase may further include one or more previously described mutations. Previously described mutations are those known to confer beneficial properties in at least one amylase having a similar fold and/or having 60% or greater amino acid sequence identity to Bacillus amylases, or in any amylase that has heretofore been referred to as “Termamyl-like.”

Furthermore, the present amylases may include any number of conservative amino acid substitutions, at positions not specifically mutated. Exemplary conservative amino acid substitutions are listed in the Table E

TABLE E
Conservative amino acid substitutions

| For Amino Acid | Code | Replace with any of |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, |
| | | Val, D-Val |
| Phenylalanine | F | D-Phe, Tyr, D-Thr, L-Dopa, His, D-His, Trp, |
| | | D-Trp, Trans-3, 4, or 5-phenylproline, |
| | | cis-3, 4, or 5-phenylproline |
| Proline | P | D-Pro, L-I-thioazolidine-4-carboxylic acid, D- |
| | | or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

The present amylases may be “precursor,” “immature,” or “full-length,” in which case they include a signal sequence, or “mature,” in which case they lack a signal sequence. Mature forms of the polypeptides are generally the most useful. Unless otherwise noted, the amino acid residue numbering used herein refers to the mature forms of the respective amylase polypeptides. The present amylase polypeptides may also be truncated to remove the N or C-termini (as exemplified by SEQ ID NO: 2), so long as the resulting polypeptides retain amylase activity.

The present amylases may be “chimeric” or “hybrid” polypeptides, in that they include at least a portion of a first amylase polypeptide, and at least a portion of a second amylase polypeptide (such chimeric amylases have recently been “rediscovered” as domain-swap amylases). The present amylases may further include heterologous signal sequence, an epitope to allow tracking or purification, or the like. Exemplary heterologous signal sequences are from B. licheniformis amylase (LAT), B. subtilis (AmyE or AprE), and Streptomyces CelA.

In another aspect, nucleic acids encoding any of the described amylase polypeptides are provided. The nucleic acid may encode a particular amylase polypeptide, or an amylase having a specified degree of amino acid sequence identity to the particular amylase. In one example, the nucleic acid encodes an amylase having at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or even at least 99% homology/identity to a reference amylase. It will be appreciated that due to the degeneracy of the genetic code, a plurality of nucleic acids may encode the same polypeptide.

The nucleic acid may also have a specified degree of homology to an exemplary polynucleotide encoding an α-amylase polypeptide. For example, the nucleic acid may have at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or even at least 99% nucleotide sequence identity to the exemplary sequence. In another example, the nucleic acid hybridizes under stringent or very stringent conditions to the exemplary sequence. Such conditions are described here but are also well known in the art. Indeed, in some embodiments, the parent enzyme is encoded by a nucleic acid sequence that hybridizes under stringent or very stringent conditions to a nucleic acid encoding Bacillus sp. strain TS-23 amylase, as exemplified by SEQ ID NO: 1 or 2.

Nucleic acids may encode a “full-length” (“fl” or “FL”) amylase, which includes a signal sequence, only the mature form of an amylase, which lacks the signal sequence, or a truncated form of an amylase, which lacks the N or C-terminus of the mature form.

A nucleic acid that encodes a α-amylase can be operably linked to various promoters and regulators in a vector suitable for expressing the α-amylase in host cells. Exemplary promoters are from B. licheniformis amylase (LAT), B. subtilis (AmyE or AprE), and Streptomyces CelA. Such a nucleic acid can also be linked to other coding sequences, e.g., to encode a chimeric polypeptide,

### 3. METHOD OF PRODUCING AND PURIFYING PROTEINS

An aspect of the present compositions and method is that the amylases can be expressed as secreted polypeptides. Methods of producing and purifying proteins that are secreted in to the culture medium from Bacillus are known in the art, as are suitable host cells for producing amylases. Exemplary methods for producing the amylases are disclosed below.

3.1 Materials and Methods for Producing Amylases

A polypeptide can be expressed using an expression vector which will typically includes control sequences including a suitable promoter, operator, ribosome binding site, translation initiation signal, and, optionally, a repressor gene or various activator genes. A large number of vectors are commercially available for use with recombinant DNA procedures, and the choice of vector will often depend on the host cell into which it is to be introduced. The vector may be an autonomously replicating vector, i.e., a vector that exists as an extrachromosomal entity, the replication of which is independent of chromosomal replication, e.g., a plasmid, a bacteriophage or an extrachromosomal element, mini-chromosome or an artificial chromosome. Alternatively, the vector may be one which, when introduced into an isolated host cell, is integrated into the host cell genome and replicated together with the chromosome(s) into which it has been integrated. The integrated gene may also be amplified to create multiple copies of the gene in the chromosome by use of an amplifiable construct driven by antibiotic selection or other selective pressure, such as an essential regulatory gene or by complementation through dose effect of an essential metabolic pathway gene.

In the vector, the DNA sequence should be operably linked to a suitable promoter sequence. The promoter may be any DNA sequence that shows transcriptional activity in the host cell of choice and may be derived from genes encoding proteins either homologous or heterologous to the host cell. Exemplary promoters for directing the transcription of the DNA sequence encoding an amylase, especially in a bacterial host, are the promoter of the lac operon of E. coli, the Streptomyces coelicolor agarase gene dagA or celA promoters, the promoters of the Bacillus licheniformis α-amylase gene (amyL), the promoters of the Bacillus stearothermophilus maltogenic amylase gene (amyM), the promoters of the Bacillus amyloliquefaciens α-amylase (amyQ), the promoters of the Bacillus subtilis xylA and xylB genes etc. For transcription in a fungal host, examples of useful promoters are those derived from the gene encoding Aspergillus oryzae TAKA amylase, Rhizomucor miehei aspartic proteinase, Aspergillus niger neutral α-amylase, A. niger acid stable α-amylase, A. niger glucoamylase, Rhizomucor miehei lipase, A. oryzae alkaline protease, A. oryzae triose phosphate isomerase, or A. nidulans acetamidase. When a gene encoding an amylase is expressed in a bacterial species such as E. coli, a suitable promoter can be selected, for example, from a bacteriophage promoter including a T7 promoter and a phage lambda promoter. Examples of suitable promoters for the expression in a yeast species include but are not limited to the Gal 1 and Gal 10 promoters of Saccharomyces cerevisiae and the Pichia pastoris AOX1 or AOX2 promoters. For expression in Trichoderma reesei, the CBHII (cellobiohydrolase II) promoter may be used

An expression vector may also comprise a suitable transcription terminator and, in eukaryotes, polyadenylation sequences operably linked to the DNA sequence encoding an α-amylase. Termination and polyadenylation sequences may suitably be derived from the same sources as the promoter.

The vector may further comprise a DNA sequence enabling the vector to replicate in the host cell. Examples of such sequences are the origins of replication of plasmids pUC19, pACYC177, pUB110, pE194, pAMB1, and pIJ702.

The vector may also comprise a selectable marker, e.g., a gene the product of which complements a defect in the isolated host cell, such as the dal genes from B. subtilis or B. licheniformis, or a gene that confers antibiotic resistance such as, e.g., ampicillin, kanamycin, chloramphenicol or tetracycline resistance. Furthermore, the vector may comprise Aspergillus selection markers such as amdS, argB, niaD and xxsC, a marker giving rise to hygromycin resistance, or the selection may be accomplished by co-transformation, such as known in the art. See e.g., International PCT Application WO 91/17243.

As noted above, while intracellular expression or solid-state fermentation may be advantageous in some respects, e.g., when using certain bacteria or fungi as host cells, one aspect of the compositions and methods contemplates expression of an α-amylase into the culture medium.

In general, “full-length,” “mature,” or “precursor” amylases includes a signal sequence at the amino terminus that permits secretion into the culture medium. If desirable, this signal peptide may be replaced by a different sequence, conveniently accomplished by substitution of the DNA sequences encoding the respective signal polypeptide.

The expression vector typically includes the components of a cloning vector, such as, for example, an element that permits autonomous replication of the vector in the selected host organism and one or more phenotypically detectable markers for selection purposes. The expression vector normally comprises control nucleotide sequences such as a promoter, operator, ribosome binding site, translation initiation signal and optionally, a repressor gene or one or more activator genes. Additionally, the expression vector may comprise a sequence coding for an amino acid sequence capable of targeting the amylase to a host cell organelle such as a peroxisome, or to a particular host cell compartment. Such a targeting sequence includes but is not limited to the sequence, SKL. For expression under the direction of control sequences, the nucleic acid sequence of the amylase is operably linked to the control sequences in proper manner with respect to expression.

The procedures used to ligate the DNA construct encoding an amylase, the promoter, terminator and other elements, respectively, and to insert them into suitable vectors containing the information necessary for replication, are well known to persons skilled in the art (see, e.g., Sambrook et al., MOLECULAR CLONING: A LABORATORY MANUAL, 2^{nd} ed., Cold Spring Harbor, 1989, and 3^{rd} ed., 2001).

An isolated cell, either comprising a DNA construct or an expression vector, is advantageously used as a host cell in the recombinant production of an amylase. The cell may be transformed with the DNA construct encoding the enzyme, conveniently by integrating the DNA construct (in one or more copies) in the host chromosome. This integration is generally considered to be an advantage, as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector as described above in connection with the different types of host cells.

Examples of suitable bacterial host organisms are Gram positive bacterial species such as Bacillaceae including Bacillus subtilis, Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus lautus, Bacillus megaterium, and Bacillus thuringiensis; Streptomyces species such as Streptomyces murinus; lactic acid bacterial species including Lactococcus sp. such as Lactococcus lactis; Lactobacillus sp. including Lactobacillus reuteri; Leuconostoc sp.; Pediococesis sp.; and Streptococcus sp. Alternatively, strains of a Gram negative bacterial species belonging to Enterobacteriaceae including E. coli, or to Pseudomonadaceae can be selected as the host organism.

A suitable yeast host organism can be selected from the biotechnologically relevant yeasts species such as but not limited to yeast species such as Pichia sp., Hansenula sp., or Kluyveromyces, Yarrowinia, Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism. Alternatively, the host organism can be a Hansenula species. Suitable host organisms among filamentous fungi include species of Aspergillus, e.g., Aspergillus niger, Aspergillus oryzae, Aspergillus tubigensis, Aspergillus awamori, or Aspergillus nidulans, Alternatively, strains of a Fusarium species, e.g., Fusarium oxysporum or of a Rhizomucor species such as Rhizomucor miehei can be used as the host organism. Other suitable strains include Thermomyces and Mucor species. In addition, Trichoderma reesei can be used as a host. A suitable procedure for transformation of Aspergillus host cells includes, for example, that described in EP 238023.

In a yet further aspect, a method of producing an α-amylase is provided comprising cultivating a host cell as described above under conditions conducive to the production of the enzyme and recovering the enzyme from the cells and/or culture medium.

The medium used to cultivate the cells may be any conventional medium suitable for growing the host cell in question and obtaining expression of an amylase. Suitable media and media components are available from commercial suppliers or may be prepared according to published recipes (e.g., as described in catalogues of the American Type Culture Collection).

In one aspect, an enzyme secreted from the host cells is used in a whole broth preparation. In the present methods, the preparation of a spent whole fermentation broth of a recombinant microorganism can be achieved using any cultivation method known in the art resulting in the expression of an alpha-amylase. Fermentation may, therefore, be understood as comprising shake flask cultivation, small- or large-scale fermentation (including continuous, batch, fed-batch, or solid state fermentations) in laboratory or industrial fermenters performed in a suitable medium and under conditions allowing the amylase to be expressed or isolated. The term “spent whole fermentation broth” is defined herein as unfractionated contents of fermentation material that includes culture medium, extracellular proteins (e.g., enzymes), and cellular biomass. It is understood that the term “spent whole fermentation broth” also encompasses cellular biomass that has been lysed or permeabilized using methods well known in the art.

An enzyme secreted from the host cells may conveniently be recovered from the culture medium by well-known procedures, including separating the cells from the medium by centrifugation or filtration, and precipitating proteinaceous components of the medium by means of a salt such as ammonium sulfate, followed by the use of chromatographic procedures such as ion exchange chromatography, affinity chromatography, or the like.

An aspect contemplates the polynucleotide in a vector is operably linked to a control sequence that is capable of providing for the expression of the coding sequence by the host cell, i.e. the vector is an expression vector. The control sequences may be modified, for example by the addition of further transcriptional regulatory elements to make the level of transcription directed by the control sequences more responsive to transcriptional modulators. The control sequences may in particular comprise promoters.

Host cells may be cultured under suitable conditions that allow expression of an amylase. Expression of the enzymes may be constitutive such that they are continually produced, or inducible, requiring a stimulus to initiate expression. In the case of inducible expression, protein production can be initiated when required by, for example, addition of an inducer substance to the culture medium, for example dexamethasone or IPTG or Sopharose. Polypeptides can also be produced recombinantly in an in vitro cell-free system, such as the TNT™ (Promega) rabbit reticulocyte system.

An amylase-expressing host also can be cultured in the appropriate medium for the host, under aerobic conditions. Shaking or a combination of agitation and aeration can be provided, with production occurring at the appropriate temperature for that host, e.g., from about 25° C. to about 75° C. (e.g., 30° C. to 45° C.), depending on the needs of the host and production of the desired amylase. Culturing can occur from about 12 to about 100 hours or greater (and any hour value there between, e.g., from 24 to 72 hours). Typically, the culture broth is at a pH of about 5.5 to about 8.0, again depending on the culture conditions needed for the host relative to production of an amylase.

3.2 Materials and Methods for Protein Purification

Fermentation, separation, and concentration techniques are well-known in the art and conventional methods can be used in order to prepare a concentrated amylase polypeptide-containing solution.

After fermentation, a fermentation broth is obtained, the microbial cells and various suspended solids, including residual raw fermentation materials, are removed by conventional separation techniques in order to obtain an amylase solution. Filtration, centrifugation, microfiltration, rotary vacuum drum filtration, ultrafiltration, centrifugation followed by ultra-filtration, extraction, or chromatography, or the like, are generally used.

It is desirable to concentrate an α-amylase polypeptide-containing solution in order to optimize recovery. Use of unconcentrated solutions requires increased incubation time in order to collect the purified enzyme precipitate.

The enzyme containing solution is concentrated using conventional concentration techniques until the desired enzyme level is obtained. Concentration of the enzyme containing solution may be achieved by any of the techniques discussed herein. Exemplary methods of purification include but are not limited to rotary vacuum filtration and/or ultrafiltration.

The enzyme solution is concentrated into a concentrated enzyme solution until the enzyme activity of the concentrated amylase polypeptide-containing solution is at a desired level.

Concentration may be performed using, e.g., a precipitation agent, such as a metal halide precipitation agent. Metal halide precipitation agents include but are not limited to alkali metal chlorides, alkali metal bromides and blends of two or more of these metal halides. Exemplary metal halides include sodium chloride, potassium chloride, sodium bromide, potassium bromide and blends of two or more of these metal halides. The metal halide precipitation agent, sodium chloride, can also be used as a preservative.

The metal halide precipitation agent is used in an amount effective to precipitate the α-amylase polypeptide. The selection of at least an effective amount and an optimum amount of metal halide effective to cause precipitation of the enzyme, as well as the conditions of the precipitation for maximum recovery including incubation time, pH, temperature and concentration of enzyme, will be readily apparent to one of ordinary skill in the art, after routine testing.

Generally, at least about 5% w/v (weight/volume) to about 25% w/v of metal halide is added to the concentrated enzyme solution, and usually at least 8% w/v. Generally, no more than about 25% w/v of metal halide is added to the concentrated enzyme solution and usually no more than about 20% w/v. The optimal concentration of the metal halide precipitation agent will depend, among others, on the nature of the specific amylase polypeptide and on its concentration in the concentrated enzyme solution.

Another alternative to effect precipitation of the enzyme is to use organic compounds. Exemplary organic compound precipitating agents include: 4-hydroxybenzoic acid, alkali metal salts of 4-hydroxybenzoic acid, alkyl esters of 4-hydroxybenzoic acid, and blends of two or more of these organic compounds. The addition of said organic compound precipitation agents can take place prior to, simultaneously with or subsequent to the addition of the metal halide precipitation agent, and the addition of both precipitation agents, organic compound and metal halide, may be carried out sequentially or simultaneously.

Generally, the organic precipitation agents are selected from the group consisting of alkali metal salts of 4-hydroxybenzoic acid, such as sodium or potassium salts, and linear or branched alkyl esters of 4-hydroxybenzoic acid, wherein the alkyl group contains from 1 to 12 carbon atoms, and blends of two or more of these organic compounds. The organic compound precipitation agents can be, for example, linear or branched alkyl esters of 4-hydroxybenzoic acid, wherein the alkyl group contains from 1 to 10 carbon atoms, and blends of two or more of these organic compounds. Exemplary organic compounds are linear alkyl esters of 4-hydroxybenzoic acid, wherein the alkyl group contains from 1 to 6 carbon atoms, and blends of two or more of these organic compounds. Methyl esters of 4-hydroxybenzoic acid, propyl esters of 4-hydroxybenzoic acid, butyl ester of 4-hydroxybenzoic acid, ethyl ester of 4-hydroxybenzoic acid and blends of two or more of these organic compounds can also be used. Additional organic compounds also include but are not limited to 4-hydroxybenzoic acid methyl ester (named methyl PARABEN), 4-hydroxybenzoic acid propyl ester (named propyl PARABEN), which also are both amylase preservative agents. For further descriptions, see, e.g., U.S. Pat. No. 5,281,526.

Addition of the organic compound precipitation agent provides the advantage of high flexibility of the precipitation conditions with respect to pH, temperature, amylase polypeptide concentration, precipitation agent concentration, and time of incubation.

The organic compound precipitation agent is used in an amount effective to improve precipitation of the enzyme by means of the metal halide precipitation agent. The selection of at least an effective amount and an optimum amount of organic compound precipitation agent, as well as the conditions of the precipitation for maximum recovery including incubation time, pH, temperature and concentration of enzyme, will be readily apparent to one of ordinary skill in the art, in light of the present disclosure, after routine testing.

Generally, at least about 0.01% w/v of organic compound precipitation agent is added to the concentrated enzyme solution and usually at least about 0.02% w/v. Generally, no more than about 0.3% w/v of organic compound precipitation agent is added to the concentrated enzyme solution and usually no more than about 0.2% w/v.

The concentrated polypeptide solution, containing the metal halide precipitation agent, and the organic compound precipitation agent, can be adjusted to a pH, which will, of necessity, depend on the enzyme to be purified. Generally, the pH is adjusted at a level near the isoelectric point of the amylase. The pH can be adjusted at a pH in a range from about 2.5 pH units below the isoelectric point (pI) up to about 2.5 pH units above the isoelectric point.

The incubation time necessary to obtain a purified enzyme precipitate depends on the nature of the specific enzyme, the concentration of enzyme, and the specific precipitation agent(s) and its (their) concentration. Generally, the time effective to precipitate the enzyme is between about 1 to about 30 hours; usually it does not exceed about 25 hours. In the presence of the organic compound precipitation agent, the time of incubation can still be reduced to less about 10 hours and in most cases even about 6 hours.

Generally, the temperature during incubation is between about 4° C. and about 50° C. Usually, the method is carried out at a temperature between about 10° C. and about 45° C. (e.g., between about 20° C. and about 40° C.). The optimal temperature for inducing precipitation varies according to the solution conditions and the enzyme or precipitation agent(s) used.

The overall recovery of purified enzyme precipitate, and the efficiency with which the process is conducted, is improved by agitating the solution comprising the enzyme, the added metal halide and the added organic compound. The agitation step is done both during addition of the metal halide and the organic compound, and during the subsequent incubation period. Suitable agitation methods include mechanical stirring or shaking, vigorous aeration, or any similar technique.

After the incubation period, the purified enzyme is then separated from the dissociated pigment and other impurities and collected by conventional separation techniques, such as filtration, centrifugation, microfiltration, rotary vacuum filtration, ultrafiltration, press filtration, cross membrane microfiltration, cross flow membrane microfiltration, or the like. Further purification of the purified enzyme precipitate can be obtained by washing the precipitate with water. For example, the purified enzyme precipitate is washed with water containing the metal halide precipitation agent, or with water containing the metal halide and the organic compound precipitation agents.

During fermentation, an α-amylase polypeptide accumulates in the culture broth. For the isolation and purification of the desired amylase, the culture broth is centrifuged or filtered to eliminate cells, and the resulting cell-free liquid is used for enzyme purification. In one embodiment, the cell-free broth is subjected to salting out using ammonium sulfate at about 70% saturation; the 70% saturation-precipitation fraction is then dissolved in a buffer and applied to a column such as a Sephadex G-100 column, and eluted to recover the enzyme-active fraction. For further purification, a conventional procedure such as ion exchange chromatography may be used.

Purified enzymes are useful for laundry and cleaning applications. For example, they can be used in laundry detergents and spot removers. They can be made into a final product that is either liquid (solution, slurry) or solid (granular, powder).

A more specific example of purification, is described in Sumitani, J. et al. (2000) “New type of starch-binding domain: the direct repeat motif in the C-terminal region of Bacillus sp. 195 α-amylase contributes to starch binding and raw starch degrading,” Biochem. J. 350: 477-484, and is briefly summarized here. The enzyme obtained from 4 liters of a Streptomyces lividans TK24 culture supernatant was treated with (NH₄)₂SO₄ at 80% saturation. The precipitate was recovered by centrifugation at 10,000×g (20 min. and 4° C.) and re-dissolved in 20 mM Tris/HCl buffer (pH 7.0) containing 5 mM CaCl₂. The solubilized precipitate was then dialyzed against the same buffer. The dialyzed sample was then applied to a Sephacryl S-200 column, which had previously been equilibrated with 20 mM Tris/HCl buffer, (pH 7.0), 5 mM CaCl₂, and eluted at a linear flow rate of 7 mL/hr with the same buffer. Fractions from the column were collected and assessed for activity as judged by enzyme assay and SDS-PAGE. The protein was further purified as follows. A Toyopearl HW55 column (Tosoh Bioscience, Montgomeryville, Pa.; Cat. No. 19812) was equilibrated with 20 in M Tris/HCl buffer (pH 7.0) containing 5 mM CaCl₂ and 1.5 M (NH₄)₂SO₄. The enzyme was eluted with a linear gradient of 1.5 to 0 M (NH₄)₂SO₄ in 20 mM Tris/HCl buffer, pH 7.0 containing 5 mM CaCl₂. The active fractions were collected, and the enzyme precipitated with (NH₄)₂SO₄ at 80% saturation. The precipitate was recovered, re-dissolved, and dialyzed as described above. The dialyzed sample was then applied to a Mono Q HR5/5 column (Amersham Pharmacia; Cat. No. 17-5167-01) previously equilibrated with 20 mM Tris/HCl buffer (pH 7.0) containing 5 mM CaCl₂, at a flow rate of 60 mL/hour. The active fractions are collected and added to a 1.5 M (NH₄)₂SO₄ solution. The active enzyme fractions were re-chromatographed on a Toyopearl HW55 column, as before, to yield a homogeneous enzyme as determined by SDS-PAGE. See Sumitani, J. et al. (2000) Biochem. J. 350: 477-484, for general discussion of the method and variations thereon.

For production scale recovery, an amylase polypeptide can be partially purified as generally described above by removing cells via flocculation with polymers. Alternatively, the enzyme can be purified by microfiltration followed by concentration by ultrafiltration using available membranes and equipment. However, for some applications, the enzyme does not need to be purified, and whole broth culture can be lysed and used without farther treatment. The enzyme can then be processed, for example, into granules.

### 4. CLEANING COMPOSITIONS

An aspect of the present compositions and methods is a cleaning composition that includes an amylase polypeptide as a component. An amylase polypeptide can be used as a component in detergent compositions for hand washing, laundry washing, dishwashing, and other hard-surface cleaning. Preferably, an amylase polypeptide is incorporated into detergents at or near a concentration conventionally used for amylase in detergents. For example, an amylase polypeptide may be added in amount corresponding to 0.00001-1 mg (calculated as pure enzyme protein) of amylase per liter of wash/dishwash liquor. Exemplary formulations are provided herein, as exemplified by the following:

4.1 Laundry Detergent Composition

An amylase polypeptide may be a component of a detergent composition, as the only enzyme or with other enzymes including other amylolytic enzymes. As such, it may be included in the detergent composition in the form of a non-dusting granulate, a stabilized liquid, or a protected enzyme. Non-dusting granulates may be produced, e.g., as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1,000 to 20,000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in, for example, GB 1483591. Liquid enzyme preparations may, for instance, be stabilized by adding a polyol such as propylene glycol, a sugar or sugar alcohol, lactic acid or boric acid according to established methods. Other enzyme stabilizers are known in the art. Protected enzymes may be prepared according to the method disclosed in for example EP 238 216. Polyols have long been recognized as stabilizers of proteins, as well as improving protein solubility.

The detergent composition may be in any useful form, e.g., as powders, granules, pastes, or liquid. A liquid detergent may be aqueous, typically containing up to about 70% of water and 0% to about 30% of organic solvent. It may also be in the form of a compact gel type containing only about 30% water.

The detergent composition comprises one or more surfactants, each of which may be anionic, nonionic, cationic, or zwitterionic. The detergent will usually contain 0% to about 50% of anionic surfactant, such as linear alkylbenzenesulfonate (LAS); α-olefinsulfonate (AOS); alkyl sulfate (fatty alcohol sulfate) (AS); alcohol ethoxysulfate (AEOS or AES); secondary alkanesulfonates (SAS); α-sulfo fatty acid methyl esters; alkyl- or alkenylsuccinic acid; or soap. The composition may also contain 0% to about 40% of nonionic surfactant such as alcohol ethoxy late (AEO or AE), carboxylated alcohol ethoxylates, nonylphenol ethoxylate, alkylpolyglycoside, alkyldimethylamineoxide, ethoxylated fatty acid monoethanolamide, fatty acid monoethanolamide, or polyhydroxy alkyl fatty acid amide (as described for example in WO 92/06154).

The detergent composition may additionally comprise one or more other enzymes, such as lipase, another amylolytic enzyme, cutinase, protease, cellulase, peroxidase, and/or laccase in any combination.

The detergent may contain about 1% to about 65% of a detergent builder or complexing agent such as zeolite, diphosphate, triphosphate, phosphonate, citrate, nitrilotriacetic acid (NTA), ethylenediaminetetraacetic acid (EDTA), diethylenetriaminepentaacetic acid (DTMPA), alkyl- or alkenylsuccinic acid, soluble silicates or layered silicates (e.g., SKS-6 from Hoechst). The detergent may also be unbuilt, i.e. essentially free of detergent builder. The enzymes can be used in any composition compatible with the stability of the enzyme. Enzymes generally can be protected against deleterious components by known forms of encapsulation, for example, by granulation or sequestration in hydro gels. Enzymes, and specifically amylases, either with or without starch binding domains, can be used in a variety of compositions including laundry and dishwashing applications, surface cleaners, as well as in compositions for ethanol production from starch or biomass.

The detergent may comprise one or more polymers. Examples include carboxymethylcellulose (CMC), poly(vinylpyrrolidone) (PVP), polyethyleneglycol (PEG), poly(vinyl alcohol) (PVA), polycarboxylates such as polyacrylates, maleic/acrylic acid copolymers and lauryl methacrylate/acrylic acid copolymers.

The detergent may contain a bleaching system, which may comprise a H₂O₂ source such as perborate or percarbonate, which may be combined with a peracid-forming bleach activator such as tetraacetylethylenediamine (TAED) or nonanoyloxybenzenesulfonate (NOBS). Alternatively, the bleaching system may comprise peroxyacids (e.g., the amide, imide, or sulfone type peroxyacids). The bleaching system can also be an enzymatic bleaching system, for example, perhydrolase, such as those described in US patent documents US2008145353, U.S. Pat. No. 7,754,460, U.S. Pat. No. 7,951,566, U.S. Pat. No. 7,723,083, and U.S. Pat. No. 8,062,875.

The enzymes of the detergent composition may be stabilized using conventional stabilizing agents, e.g., a polyol such as propylene glycol or glycerol; a sugar or sugar alcohol; lactic acid; boric acid or a boric acid derivative such as, e.g., an aromatic borate ester; and the composition may be formulated as described in, e.g., WO 92/19709 and WO 92/19708.

The detergent may also contain other conventional detergent ingredients such as e.g., fabric conditioners including clays, foam boosters, suds suppressors, anti-corrosion agents, soil-suspending agents, anti-soil redeposition agents, dyes, bactericides, tarnish inhibiters, optical brighteners, or perfumes.

The pH (measured in aqueous solution at use concentration) is usually neutral or alkaline, e.g., pH about 7.0 to about 11.0.

Particular forms of detergent compositions comprising an α-amylase can be formulated to include:

1) A detergent composition formulated as a granulate having a bulk density of at least 600 g/L comprising linear alkylbenzenesulfonate (calculated as acid) about 7% to about 12%; alcohol ethoxysulfate (e.g., C₁₂₋₁₈ alcohol, 1-2 ethylene oxide (EO)) or alkyl sulfate (e.g., C₁₆₋₁₈) about 1% to about 4%; alcohol ethoxylate (e.g., C₁₄₋₁₅ alcohol, 7 EO) about 5% to about 9%; sodium carbonate (e.g., Na₂CO₃) about 14% to about 20%; soluble silicate (e.g., Na₂O, 2SiO₂) about 2 to about 6%; zeolite (e.g., NaAlSiO₄) about 15% to about 22%; sodium sulfate (e.g., Na₂SO₄) 0% to about 6%; sodium citrate/citric acid (e.g., C₆H₅Na₃O₇/C₆H₈O₇) about 0% to about 15%; sodium perborate (e.g., NaBO₃H₂O) about 11% to about 18%; TAED about 2% to about 6%; carboxymethylcellulose (CMC) and 0% to about 2%; polymers (e.g., maleic/acrylic acid, copolymer, PVP, PEG) 0-3%; enzymes (calculated as pure enzyme) 0.0001-0.1% protein; and minor ingredients (e.g., suds suppressors, perfumes, optical brightener, photobleach) 0-5%.

2) A detergent composition formulated as a granulate having a bulk density of at least 600 g/L comprising linear alkylbenzenesulfonate (calculated as acid) about 6% to about 11%; alcohol ethoxysulfate C₁₂₋₁₈ alcohol, 1-2 EO) or alkyl sulfate (e.g., C₁₆₋₁₈) about 1% to about 3%; alcohol ethoxylate (e.g., C₁₄₋₁₅ alcohol, 7 EO) about 5% to about 9%; sodium carbonate (e.g., Na₂CO₃) about 15% to about 21%; soluble silicate (e.g., Na₂O, 2SiO₂) about 1% to about 4%; zeolite (e.g., NaAlSiO₄) about 24% to about 34%; sodium sulfate (e.g., Na₂SO₄) about 4% to about 10%; sodium citrate/citric acid (e.g., C₆H₅Na₃O₇/C₆H₈O₇) 0% to about 15%; carboxymethylcellulose (CMC) 0% to about 2%; polymers (e.g., maleic/acrylic acid copolymer, PVP, PEG) 1-6%; enzymes (calculated as pure enzyme protein) 0.0001-0.1%; minor ingredients (e.g., suds suppressors, perfume) 0-5%.

3) A detergent composition formulated as a granulate having a bulk density of at least 600 g/L comprising linear alkylbenzenesulfonate (calculated as acid) about 5% to about 9%; alcohol ethoxylate (e.g., C₁₂₋₁₅ alcohol, 7 EO) about 7% to about 14%; Soap as fatty acid (e.g., C₁₆₋₂₂ fatty acid) about 1 to about 3%; sodium carbonate (as Na₂CO₃) about 10% to about 17%; soluble silicate (e.g., Na₂O, 2SiO₂) about 3% to about 9%; zeolite (as NaAlSiO₄) about 23% to about 33%; sodium sulfate (e.g., Na₂SO₄) 0% to about 4%; sodium perborate (e.g., NaBO₃H₂O) about 8% to about 16%; TAED about 2% to about 8%; phosphonate (e.g., EDTMPA) 0% to about 1%; carboxymethylcellulose (CMC) 0% to about 2%; polymers (e.g., maleic/acrylic acid copolymer, PVP, PEG) 0-3%; enzymes (calculated as pure enzyme protein) 0.0001-0.1%; minor ingredients (e.g., suds suppressors, perfume, optical brightener) 0-5%.

4) A detergent composition formulated as a granulate having a bulk density of at least 600 g/L comprising linear alkylbenzenesulfonate (calculated as acid) about 8% to about 12%; alcohol ethoxylate (e.g., C₁₂₋₁₅ alcohol, 7 EO) about 10% to about 25%; sodium carbonate (as Na₂CO₃) about 14% to about 22%; soluble silicate (e.g., Na₂O, 2SiO₂) about 1% to about 5%; zeolite (e.g., NaAlSiO₄) about 25% to about 35%; sodium sulfate (e.g., Na₂SO₄) 0% to about 10%; carboxymethylcellulose (CMC) 0% to about 2%; polymers (e.g., maleic/acrylic acid copolymer, PVP, PEG) 1-3%; enzymes (calculated as pure enzyme protein) 0.0001-0.1%; and minor ingredients (e.g., suds suppressors, perfume) 0-5%.

5) An aqueous liquid detergent composition comprising linear alkylbenzenesulfonate (calculated as acid) about 15% to about 21%; alcohol ethoxylate C₁₂₋₁₅ alcohol, 7 EO or C₁₂₋₁₅ alcohol, 5 EO) about 12% to about 18%; soap as fatty acid (e.g., oleic acid) about 3% to about 13%; alkenylsuccinic acid (C₁₂₋₁₄) 0% to about 13%; aminoethanol about 8% to about 18%; citric acid about 2% to about 8%; phosphonate 0% to about 3%; polymers (e.g., PVP, PEG) 0% to about 3%; borate (e.g., B₄O₇) 0% to about 2%; ethanol 0% to about 3%; propylene glycol about 8% to about 1.4%; enzymes (calculated as pure enzyme protein) 0.0001-0.1%; and minor ingredients (e.g., dispersants, suds suppressors, perfume, optical brightener) 0-5%.

6) An aqueous structured liquid detergent composition comprising linear alkylbenzenesulfonate (calculated as acid) about 15% to about 21%; alcohol ethoxylate (e.g., C₁₂₋₁₅ alcohol, 7 EO, or C₁₂₋₁₅ alcohol, 5 EO) 3-9%; soap as fatty acid (e.g., oleic acid) about 3% to about 10%; zeolite (as NaAlSiO₄) about 14% to about 22%; potassium citrate about 9% to about 18%; borate (e.g., B₄O₇) 0% to about 2%; carboxymethylcellulose (CMC) 0% to about 2%; polymers (e.g., PEG, PVP) 0% to about 3%; anchoring polymers such as, e.g., lauryl methacrylate/acrylic acid copolymer; molar ratio 25:1, MW 3800) 0% to about 3%; glycerol 0% to about 5%; enzymes (calculated as pure enzyme protein) 0.0001-0.1%; and minor ingredients (e.g., dispersants, suds suppressors, perfume, optical brighteners) 0-5%.

7) A detergent composition formulated as a granulate having a bulk density of at least 600 g/L, comprising fatty alcohol sulfate about 5% to about 10%; ethoxylated fatty acid monoethanolamide about 3% to about 9%; soap as fatty acid 0-3%; sodium carbonate (e.g., Na₂CO₃) about 5% to about 10%; Soluble silicate (e.g., Na₂O, 2SiO₂) about 1% to about 4%; zeolite (e.g., NaAlSiO₄) about 20% to about 40%; Sodium sulfate (e.g., Na₂SO₄) about 2% to about 8%; sodium perborate (e.g., NaBO₃H₂O) about 12% to about 18%; TAED about 2% to about 7%; polymers (e.g., maleic/acrylic acid copolymer, PEG) about 1% to about 5%; enzymes (calculated as pure enzyme protein) 0.0001-0.1%; and minor ingredients (e.g., optical brightener, suds suppressors, perfume) 0-5%.

A detergent composition formulated as a granulate comprising linear alkylbenzenesulfonate (calculated as acid) about 8% to about 14%; ethoxylated fatty acid monoethanolamide about 5% to about 11%; soap as fatty acid 0% to about 3%; sodium carbonate (e.g., Na₂CO₃) about 4% to about 10%; soluble silicate (Na₂O, 2SiO₂) about 1% to about 4%; zeolite (e.g., NaAlSiO₄) about 30% to about 50%; sodium sulfate (e.g., Na₂SO₄) about 3% to about 11%; sodium citrate (e.g., C₆H₅Na₃O₇) about 5% to about 12%; polymers (e.g., PVP, maleic/acrylic acid copolymer, PEG) about 1% to about 5%; enzymes (calculated as pure enzyme protein) 0.0001-0.1%; and minor ingredients (e.g., suds suppressors, perfume) 0-5%.

9) A detergent composition formulated as a granulate comprising linear alkylbenzenesulfonate (calculated as acid) about 6% to about 12%; nonionic surfactant about 1% to about 4%; soap as fatty acid about 2% to about 6%; sodium carbonate (e.g., Na₂CO₃) about 14% to about 22%; zeolite (e.g., NaAlSiO₄) about 18% to about 32%; sodium sulfate (e.g., Na₂SO₄) about 5% to about 20%; sodium citrate (e.g. C₆H₅Na₃O₇) about 3% to about 8%; sodium perborate (e.g., NaBO₃H₂O) about 4% to about 9%; bleach activator (e.g., NOBS or TAED) about 1% to about 5%; carboxymethylcellulose (CMC) 0% to about 2%; polymers (e.g., polycarboxy late or PEG) about 1% to about 5%; enzymes (calculated as pure enzyme protein) 0.0001-0.1%; and minor ingredients (e.g., optical brightener, perfume) 0-5%.

10) An aqueous liquid detergent composition comprising linear alkylbenzenesulfonate (calculated as acid) about 15% to about 23%; alcohol ethoxysulfate (e.g., C₁₂₋₁₅ alcohol, 2-3 EO) about 8% to about 15%; alcohol ethoxylate (e.g., C₁₂₋₁₅ alcohol, 7 EO, or C₁₂₋₁₅ alcohol, 5 EO) about 3% to about 9%; soap as fatty acid (e.g., lauric acid) 0% to about 3%; aminoethanol about 1% to about 5%; sodium citrate about 5% to about 10%; hydrotrope (e.g., sodium toluensulfonate) about 2% to about 6%; borate (e.g., B₄O₇) 0% to about 2%; carboxymethylcellulose 0% to about 1%; ethanol about 1% to about 3%; propylene glycol about 2% to about 5%; enzymes (calculated as pure enzyme protein) 0.0001-0.1%; and minor ingredients (e.g., polymers, dispersants, perfume, optical brighteners) 0-5%.

11) An aqueous liquid detergent composition comprising linear alkylbenzenesulfonate (calculated as acid) about 20% to about 32%; alcohol ethoxylate (e.g., C₁₂₋₁₅ alcohol, 7 EO, or C₁₂₋₁₅ alcohol, 5 EO) 6.12%; aminoethanol about 2% to about 6%; citric acid about 8% to about 14%; borate (e.g., B₄O₇) about 1% to about 3%; polymer (e.g., maleic/acrylic acid copolymer, anchoring polymer such as, e.g., lauryl methacrylate/acrylic acid copolymer) 0% to about 3%; glycerol about 3% to about 8%; enzymes (calculated as pure enzyme protein) 0.0001-0.1%; and minor ingredients (e.g., hydrotropes, dispersants, perfume, optical brighteners) 0-5%.

12) A detergent composition formulated as a granulate having a bulk density of at least 600 comprising anionic surfactant (linear alkylbenzenesulfonate, alkyl sulfate, α-olefinsulfonate, α-sulfo fatty acid methyl esters, alkanesulfonates, soap) about 25% to about 40%; nonionic surfactant (e.g., alcohol ethoxylate) about 1% to about 10%; sodium carbonate (e.g., Na₂CO₃) about 8% to about 25%; soluble silicates (e.g., Na₂O, 2SiO₂) about 5% to about 15%; sodium sulfate (e.g., Na₂SO₄) 0% to about 5%; zeolite (NaAlSiO₄) about 15% to about 28%; sodium perborate (e.g., NaBO₃.4H₂O) 0% to about 20%; bleach activator (TAED or NOBS) about 0% to about 5%; enzymes (calculated as pure enzyme protein) 0.0001-0.1%; minor ingredients (e.g., perfume, optical brighteners) 0-3%.

13) Detergent compositions as described in compositions 1)-12) supra, wherein all or part of the linear alkylbenzenesulfonate is replaced by (C₁₂-C₁₈) alkyl sulfate.

14) A detergent composition formulated as a granulate having a bulk density of at least 600 g/L comprising (C₁₂-C₁₈) alkyl sulfate about 9% to about 15%; alcohol ethoxylate about 3% to about 6%; polyhydroxy alkyl fatty acid amide about 1% to about 5%; zeolite (e.g., NaAlSiO₄) about 10% to about 20%; layered disilicate (e.g., SK56 from Hoechst) about 10% to about 20%; sodium carbonate (e.g., Na₂CO₃) about 3% to about 12%; soluble silicate (e.g., Na₂O, 2SiO₂) 0% to about 6%; sodium citrate about 4% to about 8%; sodium percarbonate about 13% to about 22%; TAED about 3% to about 8%; polymers (e.g., polycarboxylates and PVP) 0% to about 5%; enzymes (calculated as pure enzyme protein) 0.0001-0.1%; and minor ingredients (e.g., optical brightener, photobleach, perfume, suds suppressors) 0-5%,

15) A detergent composition formulated as a granulate having a bulk density of at least 600 g/L comprising (C₁₂-C₁₈) alkyl sulfate about 4% to about 8%; alcohol ethoxylate about 11% to about 15%; soap about 1% to about 4%; zeolite MAP or zeolite A about 35% to about 45%; sodium carbonate (as Na₂CO₃) about 2% to about 8%; soluble silicate (e.g., Na₂O, 2SiO₂) 0% to about 4%; sodium percarbonate about 13% to about 22%; TAED 1-8%; carboxymethylcellulose (CMC) 0% to about 3%; polymers (e.g., polycarboxylates and PVP) 0% to about 3%; enzymes (calculated as pure enzyme protein) 0.0001-0.1%; and minor ingredients (e.g., optical brightener, phosphonate, perfume) 0-3%.

16) Detergent formulations as described in 1)-15) supra, which contain a stabilized or encapsulated peracid, either as an additional component or as a substitute for already specified bleach systems.

17) Detergent compositions as described supra in 1), 3), 7), 9), and 12), wherein perborate is replaced by percarbonate.

18) Detergent compositions as described supra in 1), 3), 7), 9), 12), 14), and 15), which additionally contain a manganese catalyst. The manganese catalyst for example is one of the compounds described in “Efficient manganese catalysts for low-temperature bleaching,” Nature 369: 637-639 (1994),

19) Detergent composition formulated as a non-aqueous detergent liquid comprising a liquid nonionic surfactant such as, e.g., linear alkoxylated primary alcohol, a builder system (e.g., phosphate), an enzyme(s), and alkali. The detergent may also comprise anionic surfactant and/or a bleach system.

An amylase polypeptide may be incorporated at a concentration conventionally employed in detergents. It is at present contemplated that, in the detergent composition, the enzyme may be added in an amount corresponding to 0.00001-1.0 mg (calculated as pure enzyme protein) of amylase polypeptide per liter of wash liquor.

In another embodiment, other enzymes, such as 2,6-β-D-fructan hydrolase, can be incorporated in detergent compositions comprising an α-amylase polypeptide and used for removal/cleaning of biofilm present on household and/or industrial textile/laundry.

The detergent composition may for example be formulated as a hand (manual) or machine (automatic) laundry detergent composition, including a laundry additive composition suitable for pre-treatment of stained fabrics and a rinse added fabric softener composition, or be formulated as a detergent composition for use in general household hard surface cleaning operations, or be formulated for manual or automatic dishwashing operations.

In a specific aspect, the detergent composition can comprise 2,6-β-D-fructan hydrolase in addition to an α-amylase polypeptide, and one or more other cleaning enzymes, such as a protease, a lipase, a cutinase, a carbohydrase, a cellulase, a pectinase, a pectate lyase, a mannanase, an arabinase, a galactanase, another amylolytic enzyme, a xylanase, an oxidase, a laccase, an aryl esterase, a perhydrolase, and/or a peroxidase, and/or combinations thereof.

In general the properties of the chosen enzyme(s) should be compatible with the selected detergent, (e.g., pH-optimum, compatibility with other enzymatic and non-enzymatic ingredients, etc.), and the enzyme(s) should be present in effective amounts.

Proteases:

Suitable proteases include those of animal, vegetable or microbial origin. Chemically modified or protein engineered mutants are included, as well as naturally processed proteins. The protease may be a serine protease or a metalloprotease, an alkaline microbial protease, a trypsin-like protease, or a chymotrypsin-like protease. Examples of alkaline proteases are subtilisins, especially those derived from Bacillus, e.g., subtilisin Novo, subtilisin Carlsberg, subtilisin 309, subtilisin 147, and subtilisin 168 (see, e.g., WO 89/06279). Examples of trypsin-like proteases are trypsin (e.g., of porcine or bovine origin), and Fusarium proteases (see, e.g., WO 89/06270 and WO 94/25583). Examples of useful proteases also include but are not limited to the variants described in WO 92/19729, WO 98/20115, WO 98/20116, and WO 98/34946. Commercially available protease enzymes include but are not limited to: ALCALASE®, SAVINASE®, PRIMASE™, DURALASE™, ESPERASE®, KANNASE™, and BLAZE™ (Novo Nordisk A/S and Novozymes A/S); MAXATASE®, MAXACAL™, MAXAPEM™, PROPERASE®, PURAFECT®, PURAFECT OXP™, FN2™, and FN3™ (Danisco US Inc.). Other exemplary proteases include NprE from Bacillus amyloliquifaciens and ASP from Cellulomonas sp. strain 69134.

Lipases:

Suitable lipases include those of bacterial or fungal origin. Chemically modified, proteolytically modified, or protein engineered mutants are included. Examples of useful lipases include but are not limited to lipases from Humicola (synonym Thermomyces), e.g., from H. lanuginosa (T. lanuginosus) (see e.g., EP 258068 and EP 305216), from H. insolens (see e.g., WO 96/13580); a Pseudomonas lipase (e.g., from P. alcaligenes or P. pseudoalcaligenes; see, e.g., EP 218 272), P. cepacia (see e.g., EP 331 376), P. stutzeri (see e.g., GB 1,372,034), P. fluorescens, Pseudomonas sp. strain SD 705 (see e.g., WO 95/06720 and WO 96/27002), P. wisconsinensis (see e.g., WO 96/12012); a Bacillus lipase (e.g., from B. subtilis; see e.g., Dartois et al, Biochemica et Biophysica Acta, 1131: 253-360 (1993)), B. stearothermophilus (see e.g., JP 64/744992), or B. pumilus (see e.g., WO 91/16422). Additional lipase variants contemplated for use in the formulations include those described for example in: WO 92/05249, WO 94/01541, WO 95/35381, WO 96/00292, WO 95/30744, WO 94/25578, WO 95/14783, WO 95/22615, WO 97/04079, WO 97/07202, EP 407225, and EP 260105. Some commercially available lipase enzymes include LIPOLASE® and LIPOLASE ULTRA™ (Novo Nordisk A/S and Novozymes A/S).

Polyesterases:

Suitable polyesterases can be included in the composition, such as those described in, for example, WO 01/34899 and WO 01/14629.

Amylases:

The compositions can be combined with other amylases, such as non-production enhanced amylase. These can include commercially available amylases, such as but not limited to STAINZYME®, NATALASE®, DURAMYL®, TERMAMYL®, FUNGAMYL® and BAN™ (Novo Nordisk A/S and Novozymes A/S); RAPIDASE®, POWERASE®, and PURASTAR® (from Danisco US Inc.).

Cellulases:

Cellulases can be added to the compositions. Suitable cellulases include those of bacterial or fungal origin, Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophile and Fusarium oxysporum disclosed for example in U.S. Pat. Nos. 4,435,307; 5,648,263; 5,691,178; 5,776,757; and WO 89/09259. Exemplary cellulases contemplated for use are those having color care benefit for the textile. Examples of such cellulases are cellulases described in for example EP 0495257, EP 0531372, WO 96/11262, WO 96/29397, and WO 98/08940. Other examples are cellulase variants, such as those described in WO 94/07998; WO 98/12307; WO 95/24471; PCT/DK98/00299; EP 531315; U.S. Pat. Nos. 5,457,046; 5,686,593; and 5,763,254. Commercially available cellulases include CELLUZYME® and CAREZYME® (Novo Nordisk A/S and Novozymes A/S); CLAZINASE® and PURADAX HA® (Danisco US Inc.); and KAC-500(B)™ (Kao Corporation).

Peroxidases/Oxidases:

Suitable peroxidases/oxidases contemplated for use in the compositions include those of plant, bacterial or fungal origin. Chemically modified or protein engineered mutants are included. Examples of useful peroxidases include peroxidases from Coprinus, e.g., from C. cinereas, and variants thereof as those described in WO 93/24618, WO 95/10602, and WO 98/15257. Commercially available peroxidases include for example GUARDZYME™ (Novo Nordisk A/S and Novozymes A/S).

The detergent enzyme(s) may be included in a detergent composition by adding separate additives containing one or more enzymes, or by adding a combined additive comprising all of these enzymes. A detergent additive, i.e. a separate additive or a combined additive, can be formulated e.g., as a granulate, a liquid, a slurry, and the like. Exemplary detergent additive formulations include but are not limited to granulates, in particular non-dusting granulates, liquids, in particular stabilized liquids or slurries.

Non-dusting granulates may be produced, e.g., as disclosed in U.S. Pat. Nos. 4,106,991 and 4,661,452 and may optionally be coated by methods known in the art. Examples of waxy coating materials are polyethylene oxide) products (e.g., polyethyleneglycol, PEG) with mean molar weights of 1,000 to 20,000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in, for example, GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

The detergent composition may be in any convenient form, e.g., a bar, a tablet, a powder, a granule, a paste, or a liquid. A liquid detergent may be aqueous, typically containing up to about 70% water, and 0% to about 30% organic solvent. Compact detergent gels containing about 30% or less water are also contemplated. The detergent composition can optionally comprise one or more surfactants, which may be non-ionic, including semi-polar and/or anionic and/or cationic and/or zwitterionic. The surfactants can be present in a wide range, from about 0.1% to about 60% by weight.

When included therein the detergent will typically contain from about 1% to about 40% of an anionic surfactant, such as linear alkylbenzenesulfonate, α-olefinsulfonate, alkyl sulfate (fatty alcohol sulfate), alcohol ethoxysulfate, secondary alkanesulfonate, α-sulfo fatty acid methyl ester, alkyl- or alkenylsuccinic acid, or soap.

When included therein, the detergent will usually contain from about 0.2% to about 40% of a non-ionic surfactant such as alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl-N-alkyl derivatives of glucosamine (“glucamides”).

The detergent may contain 0% to about 65% of a detergent builder or complexing agent such as zeolite, diphosphate, triphosphate, phosphonate, carbonate, citrate, nitrilotriacetic acid, ethylenediaminetetraacetic acid (EDTA), diethylenetriaminepentaacetic acid, alkyl- or alkenylsuccinic acid, soluble silicates or layered silicates (e.g., SKS-6 from Hoechst).

The detergent may comprise one or more polymers. Exemplary polymers include carboxymethylcellulose (CMC), polyvinylpyrrolidone) (PVP), polyethylene glycol) (PEG), poly(vinyl alcohol) (PVA), poly(vinylpyridine-N-oxide), poly(vinylimidazole), polycarboxylates e.g., polyacrylates, maleic/acrylic acid copolymers), and lauryl methacrylate/acrylic acid copolymers.

The enzyme(s) of the detergent composition may be stabilized using conventional stabilizing agents, e.g., as polyol (e.g., propylene glycol or glycerol), a sugar or sugar alcohol, lactic acid, boric acid, or a boric acid derivative (e.g., an aromatic borate ester), or a phenyl boronic acid derivative (e.g., 4-formylphenyl boronic acid). The composition may be formulated as described in WO 92/19709 and WO 92/19708.

It is at present contemplated that in the detergent compositions, in particular the enzyme variants, may be added in an amount corresponding to about 0.01 to about 100 mg of enzyme protein per liter of wash liquor (e.g., about 0.05 to about 5.0 mg of enzyme protein per liter of wash liquor or 0.1 to about 1.0 mg of enzyme protein per liter of wash liquor).

4.2 Cleaning Compositions

In the detergent applications, an α-amylase polypeptide is usually used in a liquid composition containing propylene glycol. The enzyme is solubilized in, for example, propylene glycol by mixing in a 25% volume/volume propylene glycol solution containing 10% calcium chloride.

An α-amylase polypeptide thereof discussed herein can be formulated in detergent compositions for use in cleaning dishes or other cleaning compositions. These can be powders, gels, or liquids. The compositions can comprise the enzyme alone, or with other amylolytic enzymes and/or with other cleaning enzymes or bleach activating enzymes, and other components common to cleaning compositions.

Thus, a dishwashing detergent composition can comprise a surfactant. The surfactant may be anionic, non-ionic, cationic, amphoteric or a mixture of these types. The detergent can contain 0% to about 90% by weight of a non-ionic surfactant, such as low to non-foaming ethoxylated propoxylated straight-chain alcohols.

The detergent composition may contain detergent builder salts of inorganic and/or organic types. The detergent builders may be subdivided into phosphorus containing and non-phosphorus-containing types. The detergent composition usually contains about 1% to about 90% of detergent builders. Examples of phosphorus-containing inorganic alkaline detergent builders, when present, include the water-soluble salts, especially alkali metal pyrophosphates, orthophosphates, and polyphosphates. An example of phosphorus-containing organic alkaline detergent builder, when present, includes the water-soluble salts of phosphonates. Examples of non-phosphorus-containing inorganic builders, when present, include water-soluble alkali metal carbonates, borates, and silicates, as well as the various types of water-insoluble crystalline or amorphous alumino silicates, of which zeolites are the best-known representatives.

Examples of suitable organic builders include the alkali metal; ammonium and substituted ammonium; citrates; succinates; malonates; fatty acid sulphonates; carboxymethoxy succinates; ammonium polyacetates; carboxylates; polycarboxylates; aminopolycarboxylates; polyacetyl carboxylates; and polyhydroxysulponates.

Other suitable organic builders include the higher molecular weight polymers and co-polymers known to have builder properties, for example appropriate polyacrylic acid, polymaleic and polyacrylic/polymaleic acid copolymers, and their salts.

The cleaning composition may contain bleaching agents of the chlorine/bromine-type or the oxygen-type. Examples of inorganic chlorine/bromine-type bleaches are lithium, sodium or calcium hypochlorite, and hypobromite, as well as chlorinated trisodium phosphate. Examples of organic chlorine/bromine-type bleaches are heterocyclic N-bromo- and N-chloro-imides such as trichloroisocyanuric, tribronioisocyanuric, dibromoisocyanuric, and dichloroisocyanuric acids, and salts thereof with water-solubilizing cations such as potassium and sodium. Hydantoin compounds are also suitable.

The cleaning composition may contain oxygen bleaches, for example in the form of an inorganic persalt, optionally with a bleach precursor or as a peroxy acid compound. Typical examples of suitable peroxy bleach compounds are alkali metal perborates, both tetrahydrates and monohydrates, alkali metal percarbonates, persilicates, and perphosphates. Exemplary activator materials are TAED, and glycerol triacetate. Enzymatic bleach activation systems may also be present in the formulation, e.g., such as perborate or percarbonate glycerol triacetate and perhydrolase (see, e.g., WO 2005/056783).

The cleaning composition may be stabilized using conventional stabilizing agents for the enzyme(s), e.g., a polyol such as, e.g., propylene glycol, a sugar or a sugar alcohol, lactic acid, boric acid, or a boric acid derivative (e.g., an aromatic borate ester).

The cleaning composition may also contain other conventional detergent ingredients, e.g., deflocculant material, filler material, foam depressors, anti-corrosion agents, soil-suspending agents, sequestering agents, anti-soil redeposition agents, dehydrating agents, dyes, bactericides, fluorescers, thickeners, and perfumes.

Although the present compositions and methods have been described with reference to the details below, it would be understood that various modifications can be made.

4.3 Methods of Assessing Amylase Activity in Detergent Compositions

Numerous amylase cleaning assays are known in the art, including swatch and micro-swatch assays. The appended Examples describe only a few such assays.

The following numbered paragraphs further describe aspects and embodiments of the present compositions and methods:

1. In one aspect, a variant α-amylase polypeptide is provided, comprising at least one combinable mutation at a productive amino acid position; wherein: (i) the combinable mutation is a mutation that improves at least one desirable property of the variant α-amylase compared to the parental α-amylase, while not significantly decreasing either expression, activity, or stability of the variant α-amylase, compared to the parental α-amylase, (ii) the productive position is an amino acid position that can be substituted with a plurality of different amino acid residues, all of which substitutions result in a variant α-amylase that meets the requirements of (i), and (iii) the combinable mutation is listed in Table C or Table D, which uses SEQ NO: 2 for numbering.

2. In some embodiments of the variant amylase of paragraph I the combinable mutation has a performance property listed in Table A.

3. In some embodiments of the variant amylase of paragraph I the combinable mutation produces a variant wherein the minimum performance indices (PI) relative to the parental amylase for (i) protein expression, (ii) activity, (iii) microswatch activity, and (iv) detergent stability or thermostability are greater than or equal to 0.9, and in addition the PI for any one of these properties is greater than or equal to 1.0.

4. In some embodiments of the variant amylase of paragraph 1 the combinable mutation produces a variant wherein the minimum performance indices (PI) relative to the parental amylase for (i) protein expression, (ii) activity, (iii) microswatch activity, and (iv) detergent stability or thermostability are greater than or equal to 0.8, and in in addition have a PI for any one of these tests that is greater than or equal to 1.2.

5. In some embodiments of the variant amylase of paragraph 1 the combinable mutation produces a variant wherein the minimum performance indices (PI) relative to the parental amylase for (i) protein expression, (ii) activity, (iii) microswatch activity, and (iv) detergent stability or thermostability are greater than or equal to 0.5, and in in addition have a PI for any one of these tests that is greater than or equal to 1.5.

6. In some embodiments of the variant amylase of any of the preceding paragraphs the combinable mutation has a sustainability score of +++, ++++, or +++++,

7. In some embodiments of the variant amylase of any of the preceding paragraphs the combinable mutation has a sustainability score of ++++, or +++++.

8. In some embodiments of the variant amylase of any of the preceding paragraphs the combinable mutation has a sustainability score of +++++.

9. In some embodiments of the variant amylase of any of the preceding paragraphs the combinable mutation has a productivity score of 1 or 2.

10. In some embodiments of the variant amylase of any of the preceding paragraphs the parental α-amylase has at least 60% amino acid sequence identity to the amino acid sequence of SEQ ID NO: 1 or SEQ ID NO: 2.

11. In some embodiments of the variant amylase of any of the preceding paragraphs the parental α-amylase has at least 70% amino acid sequence identity to the amino acid sequence of SEQ ID NO: 1 or SEQ ID NO: 2.

12. In some embodiments of the variant amylase of any of the preceding paragraphs the parental α-amylase has at least 80% amino acid sequence identity to the amino acid sequence of SEQ ID NO: 1 or SEQ ID NO: 2.

13. In some embodiments of the variant amylase of any of the preceding paragraphs the parental α-amylase has at least 90% amino acid sequence identity to the amino acid sequence of SEQ ID NO: 1 or SEQ ID NO: 2.

14. In another aspect, a composition comprising the variant amylase of any of the preceding paragraphs is provided.

15. In some embodiments of the composition of paragraph 14, the composition is effective for removing starchy stains from laundry, dishes, or textiles.

16. In some embodiments, the composition of either of paragraphs 14 or 15 further comprises a surfactant.

17. In some embodiments, the composition of any of paragraphs 14-16 is a detergent composition,

18. In some embodiments, the composition of any of paragraphs 14-17 is a laundry detergent or a laundry detergent additive.

19. In some embodiments, the composition of any of paragraphs 14-18 is manual or automatic dishwashing detergent.

20. In another aspect, a method for removing a starchy stain or soil from a surface is provided, comprising: incubating the surface in the presence of a aqueous composition comprising an effective amount of the variant amylase of any of the claims 1-13, allowing the polypeptide to hydrolyze starch components present in the starchy stain to produce smaller starch-derived molecules that dissolve in the aqueous composition, and rinsing the surface, thereby removing the starchy stain from the surface.

21. In some embodiments of the method of paragraph 20, the aqueous composition further comprises a surfactant,

22. In some embodiments of the method of either of paragraphs 20 or 21, the surface is a textile surface.

23. In some embodiments of the method of any of paragraphs 20-22, the surface is on dishes.

24. In some embodiments of the method of any of paragraphs 20-23, the surface is a soiled hard surface.

25. In another aspect, an isolated polynucleotide encoding a polypeptide of any of paragraphs 1-13 is provided.

26. In another aspect, an expression vector comprising the polynucleotide of paragraph 25 is provided.

27. In another aspect, a host cell comprising the expression vector of paragraph 26 is provided.

In order to further illustrate the compositions and methods, and advantages thereof, the following specific examples are given with the understanding that they are illustrative rather than limiting.

### EXAMPLES

### Example 1

### Assays

In the following examples, various assays were used as set forth below for ease in reading. Any deviations from the protocols provided below are indicated in the relevant sections. In these experiments, a spectrophotometer was used to measure the absorbance of the products formed after the completion of the reactions.

A. Performance index

The performance index (PI) compares the performance or stability of the variant (measured value) and the standard enzyme (theoretical value) at the same protein concentration. In addition, the theoretical values can be calculated, using the parameters of the Langmuir equation of the standard enzyme. A performance index (PI) that is greater than 1 (PI>1) indicates improved performance by a variant as compared to the standard (e.g., wild-type Amy TS23t, SEQ ID NO: 2), while a PI of 1 (PI=1) identifies a variant that performs the same as the standard, and a PI that is less than 1 (PI<1) identifies a variant that performs worse than the standard.

### B. Protein Determination Assay

This assay was performed using filtered culture supernatant from cultures grown in 96-well micro-titer plates (MTPs) over 3 days at 37° C. with shaking at 300 rpm and 80% humidity. A fresh 96-well round-bottom MTP containing 25 μL supernatant per well was used for the High Performance Liquid Chromatography (HPLC) protein determination method. Supernatants were diluted three fold into 25 mM sodium acetate, pH 5.5 and 20 μL of each diluted sample was analyzed. An Agilent 1100 (Hewlett Packard) HPLC equipped with a Poroshell 300SB-C8 (Agilent Technologies) column was used. Sample was bound to the column using 25 mM sodium acetate, pH 5.5 and eluted over a gradient up to 70% acetonitrile. Absorbance was read at 222 nm, integrated using ChemStation software (Agilent Technologies) and the protein concentration of samples was determined based on a standard curve of purified Amy TS23t protein.

### C. Ceralpha α-Amylase Activity Assay

The Ceralpha α-amylase assay was performed using the Ceralpha HR Kit (Megazyme, Wicklow, Ireland). The assay involves incubating culture supernatant with a substrate mixture under defined conditions. The reaction is terminated (and color developed) by the addition of borate buffer (200 mM Boric acid/NaOH buffer, pH 10). The substrate used was a mixture of the defined oligosaccharide “nonreducing-end blocked p-nitrophenyl maltoheptaoside” (BPNPG7) and excess levels of α-glucosidase (which has no action on the native substrate due to the presence of the “blocking group”). On hydrolysis of the oligosaccharide by endoacting α-amylase (or G6 amylase), the excess quantities of α-glucosidase present in the mixture give instantaneous and quantitative hydrolysis of the p-nitrophenyl maltosaccharide fragment to glucose and free p-nitrophenol. The absorbance at 405 nm was measured, which relates directly to the level of amylase in the sample analysed.

The equipment used for this assay included a Biomek FX Robot (Beckman Coulter); a SpectraMAX MTP Reader (type 340-Molecular Devices) and iEMS incubator/shaker (Thermo Scientific). The reagent and solutions used were:

1) p-nitrophenyl maltoheptaoside (BPNPG7) substrate (Megazyme Ceralpha HR kit);

2) 50 mM MOPS, 0.005% TWEEN® 80 buffer, pH 7 (dilution buffer); and

3) 200 mM Boric acid/NaOH buffer, pH 10 (STOP buffer).

A vial containing 54.5 mg BPNPG7 substrate was dissolved in 10 mL of MilliQ water and then diluted into 30 mL of dilution buffer to make up 40 mL of the working substrate (1.36 mg/mL). The amylase samples (fermentation supernatant) were diluted 40× with dilution buffer. The assay was performed by adding 54 of diluted amylase solution into the wells of a MTP followed by the addition of 55 μL of diluted BPNPG7 working substrate solution. The solutions were mixed and the MTP was sealed with a plate seal and placed in an incubator/shaker (iEMS-Thermo Scientific) for 4 minutes at 25° C. The reaction was terminated by adding 70 μL STOP buffer and the absorbance was read at wavelength 400 nm in an MTP-Reader. A non-enzyme control was used to correct for background absorbance values.

### D. CS-28 Rice Starch Microswatch Assay

The principle of this amylase assay is the liberation of an orange-dye due to the hydrolysis of rice starch incorporated in a cotton microswatch. The absorbance at 488 nm of the wash liquid was measured and related to the level of amylase activity in the sample analyzed at the desired conditions (pH, temperature, and buffer).

The equipment used for this assay included a Biomek FX Robot (Beckman Coulter), a SpectraMAX MTP Reader (type 340-Molecular Devices) and iEMS incubator/shaker (Thermo Scientific). The reagent and solutions used were:
1) CS-28 Microswatches (rice starch, colored);
2) 10 mM HEPES, 2 mM CaCl₂, 0.005% TWEEN 80 buffer, pH 8.0, conductivity 5 mS/cm;
3) 10 mM HEPES, 2 mM CaCl₂, 0.005% TWEEN 80 buffer, pH 8.0, conductivity 5 mS/cm (adjusted with 5M NaCl);
3) 25 mM CAPS, 2 mM CaCl₂, 0.005% TWEEN 80 buffer, pH 10.0; conductivity 5 mS/cm (adjusted with 5M NaCl); and
4) 10 mM NaCl, 0.1 mM CaCl₂, 0.005% TWEEN 80.

CS-28 Microswatches of 5.5 mm circular diameter were provided by the Center for Testmaterials (CFT, Vlaardingen, The Netherlands). Two microswatches were placed in each well of a 96-well Corning 9017 flat bottomed polystyrene MTP. The culture supernatants were diluted in 10 mM NaCl, 0.1 mM CaCl₂, 0.005% TWEEN®80 solution to approximately 1 ppm.

The incubator/shaker was set at the desired temperature, 25° C. (room temperature) or 32° C. 171 μL of either HEPES or CAPS buffer was added to each well of microswatch containing MTP and subsequently 9 μL of diluted enzyme solution was added to each well resulting in a total volume of 180 μL/well. The MTP was sealed with a plate seal and placed in the iEMS incubator/shaker and incubated for 15 minutes at 1150 rpm at 25° C. for cleaning at pH8, both low (1 mS/cm) and high (5 mS/cm) conductivity and for 30 minutes at 1150 rpm at 32° C. for cleaning at pH 10, high conductivity (5 mS/cm). Following incubation under the appropriate conditions, 100 μL of solution from each well was transferred to a new MTP, and the absorbance at 488 nm was measured using a MTP-spectrophotometer. Controls containing two microswatches and buffer but no enzyme were included for subtraction of background cleaning performance.

The obtained absorbance value was corrected for the blank value (obtained after incubation of microswatches in the absence of enzyme), and the resulting absorbance provided a measure of the hydrolytic activity. A performance index (PI) was calculated for each sample. For the PI calculation for the wash performance indices, a curve-fit was made based on the wild-type Amy TS23t enzyme (SEQ ID NO: 2), using the Langmuir equation. Using the protein concentration of the variants, the expected performance based on the curve-fit was calculated. The observed performance was divided by the calculated performance. This value was then divided by the performance of the wild-type AmyTS23t enzyme.

### E. Thermostability Assay—Determination of Initial and Residual Activities

The thermostability of the amylase variant in relation to a reference amylase (wild-type Amy TS23t, SEQ ID NO: 2) was determined by incubating the amylase samples under defined conditions in MOPS buffer, pH 7. The temperature of the incubation was selected such that approximately 40% of the initial reference amylase activity was lost. The initial and residual amylase activities were determined using the Ceralpha α-amylase method described in section C above.

The equipment used for this assay included a Biomek FX Robot (Beckman Coulter); a SpectraMAX MTP Reader (type 340-Molecular Devices), a Tetrad2 DNA Engine PCR machine (BioRad), and iEMS incubator/shaker (Thermo Scientific). The reagent solutions used were:

1) p-nitrophenyl maltoheptaoside (BPNPG7) substrate (Megazyme Ceralpha HR kit);

2) 50 mM MOPS, 1.0 mM CaCl₂, 0.005% TWEEN®80 buffer, pH 7 (dilution buffer); and

3) 200 mM Boric acid/NaOH buffer, pH 10 (STOP buffer)

The culture supernatants were diluted 40× in dilution buffer to yield samples in a concentration range of 0-4 ppm. 5 μL of diluted sample was used to determine the initial amylase activity and 70 μL was used for heat incubation. 70 μL samples were put in each well of a 96 well PCR plate (VWR 211-0297) that was sealed with an aluminum seal and incubated at 75° C. for 4 minutes in the Tetrad2 DNA Engine PCR machine. Initial (tᵢₙᵢₜᵢₐₗ) and residual (t_{residual}) amylase activity was determined by the Ceralpha α-amylase assay as described above in Section C using a 5 μL sample.

For each variant, the ratio of the residual and initial amylase activities was used to calculate thermostability as follows: Thermostability/[t_{residual} value]/[tᵢₙᵢₜᵢₐₗ value], so the thermostability activity ratio was calculated based on enzyme activity after the heat incubation, divided by enzyme activity before the heat incubation. The performance index for thermostability was determined by dividing the activity ratio of the variant enzyme, with that of the similarly treated wild-type AmyTS23t enzyme (SEQ ID NO: 2).

### F. Detergent Stability Assay

The stability of the reference amylase and variants thereof was measured after incubation under defined conditions in the presence of 10% commercially purchased Persil Color Gel detergent, Henkel (purchased in 2011). The detergent was heat inactivated before use, and the initial and residual amylase activities were determined using the Ceralpha α-amylase assay as described in section C above.

The equipment used for this assay included a Biomek FX Robot (Beckman Coulter); a SpectraMAX MTP Reader (type 340-Molecular Devices), a Tetrad2DNA Engine PCR machine (Biorad), and iEMS incubator/shaker (Thermo Scientific). The reagent solutions used were:

1) p-nitrophenyl maltoheptaoside (BPNPG7) substrate (Megazyme Ceralpha HR kit):

2) Liquid detergent (Persil color gel, enzyme-inactivated, 2 hrs at 80° C.);

3) 50 mM MOPS, OA mM CaCl₂, 0.005% TWEEN® 80 buffer, pH 7 (dilution buffer);

4) 10% detergent solution diluted in dilution buffer;

5) 200 mM Boric acid NaOH buffer, pH 10 (STOP buffer)

6) Amylase culture supernatants containing 0-260 μg/mL protein

90 μL of a 10% detergent solution was added to a 96 well PCR plate and mixed with 10 μL of original culture supernatant. A sample from the PCR plate was diluted 3× in dilution buffer and a 5 μL aliquot of this dilution was used to determine initial amylase activity. The PCR plate was incubated in a Tetrad PCR block at 64° C. for 5 minutes. After incubation, detergent-enzyme mix was diluted 3× in dilution buffer and residual activity was measured. Initial(tᵢₙᵢₜᵢₐₗ) and residual (t_{residual}) amylase activity was determined by the Ceralpha α-amylase assay as described above in Section C using a 5 μL sample.

For each variant, the ratio of the residual and initial amylase activities was used to calculate the detergent stability as follows: Detergent stability=[t_{residual} value]/[tᵢₙᵢₜᵢₐₗ value], so the detergent stability activity ratio was calculated based on enzyme activity after the heat incubation, divided by enzyme activity before the heat incubation.

For each sample (variants) the performance index (PI) was calculated. The performance index for detergent stability was determined by comparing the detergent stability of the variant enzyme with that of the similarly treated wild-type AmyTS23t enzyme (SEQ ID NO: 2).

### Example 2

### Generation of B. subtilis Strains Expressing and Secreting Amy TS23t α-Amylase and Variants Thereof

This Example describes the construction of Bacillus subtilis strains expressing Amy TS23t and variants of Amy TS23t. The mature α-amylase from Bacillus sp. stain TS-23 (i.e., AmyTS23) has the amino acid sequence of SEQ ID NO: 1:

| | |
|---|---|
| NTAPINETMM QYFEWDLPND GTLWTKVKNE AANLSSLGIT ALWLPPAYKG | 50 |
| TSQSDVGYGV YDLYDLGEFN QKGTIRTKYG TKTQYIQAIQ AAKAAGMQVY | 100 |
| ADVVFNHKAG ADGTEFVDAV EVDPSNRNQE TSGTYQIQAW TKFDFPGRGN | 150 |
| TYSSFKWRWY HFDGTDWDES RKLNRIYKFR STGKAWDWEV DTENGNYDYL | 200 |
| MFADLDMDHP EVVTELKNWG TWYVNTTNID GFRLDAVKHI KYSFFPDWLT | 250 |
| YVRNQTGKNL FAVGEFWSYD VNKLHNYITK TNGSMSLFDA PLHNNFYTAS | 300 |
| KSSGYFDMRY LLNNTLMKDQ PSLAVTLVDN HDTQPGQSLQ SWVEPWFKPL | 350 |
| AYAFILTRQE GYPCVFYGDY YGIPKYNIPG LKSKIDPLLI ARRDYNYGTQ | 400 |
| RDYIDHQDII GWTREGIDTK PNSGLAALIT DGPGGSKWMY VGKKHAGKVF | 450 |
| YDLTGNRSDT VTINADGWGE FKVNGGSVSI WVAKTSNVTF TVNNATTTSG | 500 |
| QNVYVVANIP ELGNWNTANA IKMNPSSYPT WKATIALPQG KAIEFKFIKK | 550 |
| DQAGNVIWES TSNRTYTVPF SSTGSYTASW NVP | 583 |

A C-terminal truncated form of the mature α-amylase from Bacillus sp. strain TS-23 (i.e., AmyTS23t or BASE) has the amino acid sequence of SEQ ID NO: 2:

NTAPINETMMQYFEWDLPNDGTLWTKVKNEAANLSSLGITALWLPPAYKGTSQ
SDVGYGVYDLYDLGEFNQKGTIRTKYGTKTQYIQAIQAAKAAGMQVYADVVFN
HKAGADGTEFVDAVEVDPSNRNQETSGTYQIQAWTKFDFPGRGNTYSSFKWRW
YHFDGTDWDESRKLNRIYKFRSTGKAWDWEVDTENGNYDYLMFADLDMDHPEV
VTELKNWGTWYVNTTNIDGFRLDAVKHIKYSFFPDWLTYVRNQTGKNLFAVGE
FWSYDVNKLHNYITKTNGSMSLFDAPLHNNFYTASKSSGYFDMRYLLNNTLMK
DQPSLAVTLVDNHDTQPGQSLQSWVEPWFKPLAYAFILTRQEGYPCVFYGDYY
GIPKYNIPGLKSKIDPLLIARRDYAYGTQRDYIDHQDIIGWTREGIDTKPNSG
TAALITDGPGGSKWMYVGKKHAGKVFYDLTGNRSDTVTINADGWGEFKVNGGS
VSIWVAK

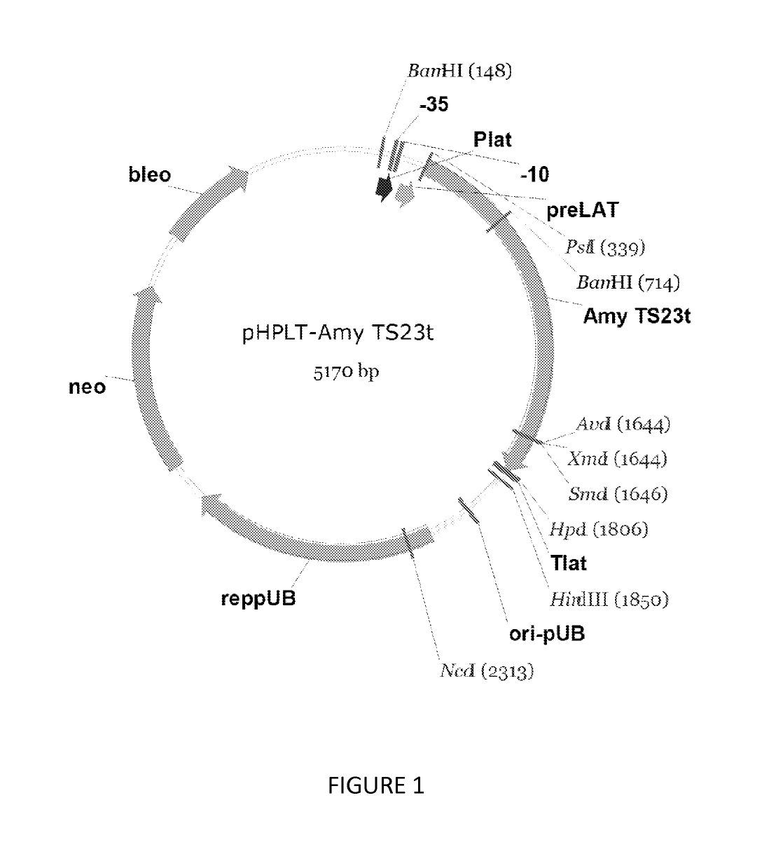

A synthetic DNA fragment (produced by GeneArt AG/Life Technologies, Im Gewerbepark B35, 93059 Regensburg, Germany), served as template DNA for the construction of Bacillus subtilis strains expressing Amy TS23t and variants of Amy TS23t. To express Amy TS23t, the Amy TS23t DNA fragment was cloned in the pHPLT vector (Solingen et al. (2001) Extremophiles 5:333-341) by GeneArt, using the unique PstI and HpaI restriction sites. The pHPLT expression vector contains the B. licheniformis LAT promoter (Plat), Lat leader sequence, and additional elements from pUB110 plasmid (McKenzie et al. (1986) Plasmid 15: 93-103) including a replicase gene (reppUB), a neomycin/kanamycin resistance gene (neo), and a bleomycin resistance marker (bless). pHPLT-Amy TS23t is shown in FIG. 1. A B. subtilis strain (ΔaprE, ΔnprE) was transformed using the pHPLT-Amy TS23t vector DNA as described in WO2002/14490, incorporated herein by reference.

The B. subtilis transformants were selected on agar plates containing Heart infusion agar (Difco, Cat. no 244400) and 10 mg/L neomycin sulfate (Sigma, Catalog No. N-1876; contains 732 μg neomycin per mg). Selective growth of B. subtilis transformants harboring the pHPLT-Amy TS23t vector was performed in shake flasks containing MBD medium (a MOPS based defined medium), 5 mM CaCl₂ and 10 mg/L neomycin. MBD medium was made essentially as known in the art (see, e.g., Neidhardt et al. (1974) J. Bacteriol. 119: 736-747), except that NH₄Cl₂, FeSO₄, and CaCl₂ were omitted from the base medium, 3 mM K₂HPO₄ was used, and the base medium was supplemented with 60 mM urea, 75 g/L glucose, and 1% soytone. The micronutrients were made up as a 100× stock solution containing in one liter, 400 mg FeSO₄ 7H₂O, 100 mg MnSO₄.H₂O, 100 mg ZnSO₄ 7H₂O, 50 mg CuCl₂ 2H₂O, 100 mg CoCl₂ 6H₂O, 100 mg NaMoO₄ 2H₂O, 100 mg Na₂B₄O₇ 10H₂O, 10 ml of 1M CaCl₂, and 10 mL of 0.5 M sodium citrate. Growth resulted in the production of secreted AmyTS23t amylase with starch hydrolyzing activity.

### Example 3

### Generation of Amy TS23t Site Evaluation Libraries (SELs)

Site Evaluation Library (SEL) production was performed by GeneArt AG using a proprietary process (WO2004/_{059556A3}). Methods and devices for optimizing a nucleotide sequence for the purpose of expression of a protein by PCR, and the manufacture of DNA molecules utilized technology owned by or licensed to GeneArt (European Patent Nos. 0 200 362 and 0 201 184; and U.S. Pat. Nos. 4,683,195, 4,683,202 and 6,472,184). The construction of Amy TS23t SELs described in this example was performed by GeneArt using their technology platform for gene optimization, gene synthesis and library generation under proprietary GeneArt know how and/or intellectual property. The sequential permutation approach of GeneArt, to produce SELs, is described in general on the company's web site (http://www.geneart.com/english/products-services/directed/evolution/sequential-permutation-libraries/index.html).

The pHPLT-Amy TS23t plasmid DNA (FIG. 1) served as template to produce the SELs. Amy TS23t SELs were produced by GeneArt at all positions pre-selected by the inventors. The accompanying DNA codons were each substituted into randomly mutated codons, coding for at least 15 different amino acids. The codon mutagenized pHPLT-Amy TS23t mixtures were used to transform competent B. subtilis cells as described (WO2002/014490), in order to generate the Amy TS23t Site Evaluation Libraries.

Transformation mixtures were plated on Heart Infusion agar plates containing 10 mg/L neomycin sulfate. For each library, single colonies were picked and grown in TSB (tryptone and soy based broth) liquid media under 10 mg/L neomycin selection for subsequent DNA isolation and gene sequence analysis. Sequence analysis data revealed a maximum of 19 Amy TS 23t mature single variants per library. To generate Amy TS 23t and Amy TS23t variant enzyme samples for biochemical characterization experiments, selective growth of the Amy TS23t SEL variant clones was performed in 96 well MTP (Costar 3599) at 37° C. for 68 hours in 200 μL MBD medium per well containing 10 mg/L neomycin and 5 mM CaCl₂.

### Example 4

### Identification of Combinable and Productive Mutations

Performance index (PI) values were determined for all the Amy TS23t amylase variants tested using the assays described in Example 1: CS-28 microswatch assay (at both pH8 and pH10), detergent stability, thermostability, and protein determination.

Productive positions are described as those positions within a molecule that are most useful for making combinatorial variants exhibiting an improved characteristic, where the position itself allows for at least one combinable mutation. Combinable mutations are mutations at any amino acid position that can be used to make combinatorial variants. Combinable mutations improve at least one desired property of the molecule, while not significantly decreasing expression, activity, or stability. Combinable mutations can be grouped as follows:

Group A:
A mutation that produces a variant wherein the minimum performance indices (PI) relative to a defined parental protein for: (i) protein expression, (ii) CS-28 microswatch activity at pH 8 (25° C.) or pH10 (32° C.), and (iii) detergent stability or thermostability are greater than or equal to 0.9, and in addition have a PI for any one of these tests that is greater than or equal to 1.0.

Group B:
A mutation that produces a variant wherein the minimum performance indices (PI) relative to a defined parental protein for: (i) protein expression, (ii) CS-28 microswatch activity at pH 8 (25° C.) or pH10 (32° C.), and (iii) detergent stability or thermostability are greater than or equal to 0.8, and in addition have a PI for any one of these tests that is greater than or equal to 12.

Group C:
A mutation that produces a variant wherein the minimum performance indices (PI) relative to a defined parental protein for: (i) protein expression, (ii) CS-28 microswatch activity at pH 8 (25° C.) or pH10 (32° C.), and (iii) detergent stability or thermostability are greater than or equal to 0.5, and in addition have a PI for any one of these tests that is greater than or equal to 1.5.

The properties of combinable mutations are summarized in Table 4.1.

TABLE 4.1
Properties for each group of combinable mutations

| | Performance Index (PI) | | | |
|---|---|---|---|---|
| | | Cleaning | Stability (detergent | Minimum PI in one |
| Group | Expression | (pH 8 or 10) | or thermal) | or more tests |
| A | ≧0.9 | ≧0.9 | ≧0.9 | X ≧ 1.0 |
| B | ≧0.8 | ≧0.8 | ≧0.8 | X ≧ 1.2 |
| C | ≧0.5 | ≧0.5 | ≧0.5 | X ≧ 1.5 |

Preferred combinable mutations are at “productive positions,” as described, below. In the case of the present amylases, “activity” refers to α-amylase activity, which can be measured as described, herein.

Productive positions are amino acid positions that are tolerant to substitution with different amino acid residues, wherein the resulting variants meet a set of performance criteria for combinability, as set forth above. Productive positions can be assigned a Productivity Score as follows:
Positions where less than 15% of the substitutions at a given position fall within groups A, B, or C are given a Productivity Score of “1”.
Positions where less than 40%, but greater than, or equal to 15% of the substitutions at a given position fall within groups A, B, or C are given a Productivity Score of “2”.
Positions where less than 75%, but greater than, or equal to 40% of the substitutions at a given position fall within groups A, B, or C are given a Productivity Score of “3”.
Positions where 75% or more of the substitutions at a given position fall within groups A, B, or C are given a Productivity Score of “4”.

Preferred productive positions are combinable mutations.

Suitability score refers to the ability of one or more combinable mutations to be used to make combinatorial variants, based on the performance criteria for combinability, (i.e., A, B, and C, as set forth, above) in which each of the mutations fall. A higher suitability score indicates a mutation or mutations that are more suitable for use in making combinatorial variants. Suitability scores are described in Table 4.2.

TABLE 4.2
Definitions of suitability scores

| Substitutions Occur in | |
|---|---|
| Group(s) | Suitability Score |
| A, B and C | +++++ |
| A and B | ++++ |
| A or (B and C) | +++ |
| B | ++ |
| C | + |

Table 4.3 shows the Productivity Score (4, 3, or 2,) calculated for each position in the Amy TS23t protein. Subsets of these positions are listed in Tables 4.4 and 4.5. No positions were calculated to have a productivity score of 1. For each Amy TS23t position, variants are listed according to the suitability score they received (+, ++, +++, ++++, or +++++). Position numbering is based on the mature Amy TS23t polypeptide (SEQ ID NO: 2).

TABLE 4.3
Productivity score for positions in the Amy TS23t protein.
VARIANTS SUITABILITY SCORE

| | Productivity | | | | | |
|---|---|---|---|---|---|---|
| POS | Score | (+) | (++) | (+++) WT AA 1^{ST} | (++++) | (+++++) |
| 1 | 2 | | | NHK | QR | |
| 2 | 2 | | A | TEQ | | |
| 3 | 2 | W | G | ADEHPT | | |
| 5 | 4 | | | IDEHKNRTVY | S | |
| 7 | 2 | R | N | EHKP | Q | |
| 12 | 2 | | S | YDHQT | G | |
| 13 | 3 | | S | FY | | |
| 15 | 2 | | AH | WY | | |
| 16 | 1 | | | DE | | |
| 17 | 4 | | | LCDEFGHIMNQRSTV | AY | |
| 18 | 2 | E | | PQ | H | |
| 19 | 3 | G | | NDEHPS | AKT | R |
| 20 | 3 | | | DN | | |
| 22 | 3 | C | DL | TAHQ | KS | |
| 23 | 4 | | | LAEFGMNSTWY | Q | |
| 25 | 3 | | | TDGLNPSW | H | |
| 26 | 2 | | | KAGHRS | N | |
| 27 | 3 | | | VEGNST | Q | |
| 28 | 3 | | D | KGHNQRST | | |
| 29 | 3 | | | NDGKPRST | AH | |
| 30 | 3 | | | ED | | |
| 31 | 3 | | | AGHNPTY | QS | |
| 32 | 1 | | | AGP | | |
| 33 | 2 | | | NDGP | | |
| 34 | 2 | | | LHNQ | | |
| 36 | 2 | | D | SPQT | | |
| 37 | 3 | | | LHIMQRSTVY | | |
| 40 | 1 | | | TR | | |
| 41 | 2 | | | ADQRS | | |
| 43 | 1 | | | WY | | |
| 44 | 1 | | | LT | | |
| 48 | 1 | | | YQT | | |
| 50 | 2 | D | | GS | A | |
| 51 | 1 | | R | T | Q | |
| 52 | 1 | | KR | S | | |
| 53 | 2 | | K | QAS | | |
| 54 | 3 | | W | SDEHKNRT | AQ | |
| 56 | 4 | | | VAEHINPQRST | | |
| 59 | 2 | CK | | GS | | |
| 60 | 2 | | IL | VQ | GT | AS |
| 63 | 2 | | S | LV | EM | |
| 64 | 1 | | | YT | S | |
| 66 | 1 | | | L | I | |
| 68 | 1 | N | | ED | | |
| 70 | 3 | | | NAHKPQRS | D | |
| 71 | 1 | | | QAS | | |
| 73 | 2 | | AH | GQR | D | |
| 74 | 1 | LW | | T | | |
| 75 | 4 | | LT | IAEGHKMRSW | FQ | |
| 79 | 1 | | | YHW | | |
| 81 | 1 | | | TH | | |
| 82 | 2 | | I | KAEFLNY | | |
| 83 | 3 | | | TADFHIKLQWY | | |
| 87 | 2 | | | QLNSTW | P | |
| 88 | 1 | | | AS | | |
| 89 | 2 | | A | IMV | | |
| 90 | 2 | | | QAGH | | |
| 91 | 3 | | E | AFGHIRW | K | |
| 92 | 1 | | | AG | | |
| 93 | 1 | | R | K | | |
| 94 | 3 | | | AFHILSTW | R | |
| 95 | 3 | | | AGLQRST | IK | |
| 96 | 2 | | K | GACE | | |
| 97 | 2 | | | MFV | | |
| 103 | 1 | | I | VA | | |
| 105 | 1 | I | | F | | |
| 106 | 3 | D | | N | | |
| 107 | 1 | CS | | H | | |
| 108 | 2 | | AL | KR | | |
| 109 | 1 | ET | | A | | |
| 110 | 1 | | | GAS | | |
| 113 | 3 | | LT | GER | AHQ | D |
| 114 | 1 | R | | TS | | |
| 116 | 2 | W | N | FAHIQR | | |
| 117 | 2 | W | AS | VEQ | P | |
| 118 | 2 | AKS | | D | | |
| 120 | 2 | AN | | V | | |
| 122 | 1 | | | V | | C |
| 123 | 1 | | | DAS | | |
| 125 | 2 | | K | SQ | FHR | |
| 126 | 1 | | | N | D | |
| 128 | 2 | C | Y | N | W | |
| 129 | 3 | | LMT | QAV | KR | I |
| 131 | 1 | | | T | | I |
| 133 | 2 | | H | GN | DP | E |
| 134 | 2 | | DS | TA | P | |
| 135 | 2 | HIKLM | | Y | | |
| 136 | 2 | CHT | AGPR | Q | | |
| 138 | 2 | CLMT | DN | Q | | |
| 139 | 1 | | | AP | | |
| 140 | 1 | | | WHK | | |
| 142 | 3 | | GHTV | KAILQSW | | |
| 144 | 1 | H | | D | | |
| 146 | 1 | | | PA | | |
| 147 | 2 | | AL | GH | | W |
| 149 | 3 | | | GAHILQRWY | D | |
| 150 | 2 | | GK | N | D | |
| 152 | 1 | S | | YH | | |
| 154 | 2 | FIV | K | SNQ | G | |
| 156 | 1 | | F | KR | | |
| 158 | 2 | | A | RKW | LS | |
| 160 | 4 | | M | YS | AEKLPQRTVW | DIN |
| 162 | 1 | M | | F | | |
| 165 | 2 | DF | C | T | | IV |
| 166 | 3 | CGHPQ | | D | | |
| 167 | 2 | ANS | L | W | | |
| 168 | 2 | N | | D | | |
| 169 | 2 | | | EFHIQT | A | |
| 170 | 4 | FL | CDM | SAQTWY | H | |
| 171 | 1 | | L | R | | |
| 172 | 3 | | | KADLNQRY | F | |
| 173 | 1 | | | LQW | | |
| 174 | 4 | | | NACDS | HQT | R |
| 175 | 2 | CL | K | R | | |
| 177 | 2 | H | | YFW | | |
| 178 | 2 | AI | | KG | | LQ |
| 179 | 1 | I | | F | | |
| 180 | 4 | CG | W | RIMV | | ADKNQT |
| 181 | 3 | CDHMPQT | | S | | AG |
| 182 | 4 | H | VY | TW | FR | ACDEGLMNPQ |
| 183 | 1 | EN | | G | | |
| 185 | 3 | C | G | ANS | Q | DE |
| 192 | 1 | I | D | T | | |
| 194 | 2 | | | NF | H | |
| 195 | 2 | M | V | GA | H | C |
| 197 | 4 | AN | F | Y | | D |
| 202 | 2 | | GH | FLMNY | S | |
| 203 | 2 | CKN | E | AD | Q | |
| 207 | 1 | | | M | Y | |
| 209 | 4 | MNQR | | HEY | A | CW |
| 210 | 4 | | LM | P | CEGHKNQT | DR |
| 212 | 1 | P | | V | | |
| 213 | 1 | | | VT | | P |
| 214 | 3 | | | TAKQRS | DE | |
| 215 | 1 | | H | E | | |
| 217 | 2 | AS | Q | K | | |
| 218 | 1 | | | NS | | |
| 221 | 4 | | EW | THNP | ADKQRSY | |
| 223 | 2 | L | S | YF | | |
| 224 | 2 | | | VKR | Q | |
| 225 | 1 | | | ND | | |
| 226 | 3 | | | TAHNQ | | |
| 228 | 3 | | | NDEFGHL | PQS | |
| 229 | 2 | | S | IV | | |
| 232 | 1 | | | FW | | |
| 234 | 2 | | GS | LIM | | A |
| 236 | 1 | E | | A | | |
| 237 | 1 | | I | V | | |
| 242 | 1 | F | | Y | | |
| 243 | 2 | A | | S | | DQ |
| 244 | 3 | | | FHV | NS | LMT |
| 245 | 4 | | T | FNSV | | AMQ |
| 246 | 3 | | F | PK | DQRS | AEY |
| 249 | 1 | | | LQ | | |
| 250 | 4 | | I | TADEGHKQRY | | |
| 251 | 2 | P | | YHMQRS | | |
| 252 | 1 | | | VL | | |
| 253 | 2 | | H | RKT | | |
| 254 | 3 | | AEH | NDKMQRT | | |
| 255 | 1 | | | QR | | |
| 256 | 2 | | DQRY | T | N | |
| 257 | 2 | | | GDKNRST | | |
| 258 | 2 | C | N | KQ | | |
| 260 | 1 | | | LM | R | |
| 262 | 2 | M | | AG | | |
| 264 | 1 | | | GS | | |
| 268 | 2 | | | SN | T | |
| 269 | 2 | EHLM | AS | Y | | |
| 271 | 4 | ADGW | HQ | VY | IPRS | MT |
| 272 | 3 | | HQ | NEMR | ADKST | |
| 273 | 1 | R | | K | | |
| 275 | 2 | D | R | HQ | | |
| 279 | 3 | | | TDHNQRS | | |
| 282 | 2 | LM | E | N | | D |
| 283 | 3 | DFT | Q | GE | AHKNRS | |
| 284 | 2 | | | SDEHKRT | Q | |
| 285 | 2 | | A | M | QTV | I |
| 286 | 2 | | | SDHQT | NR | |
| 287 | 1 | | | LI | | |
| 294 | 2 | E | | NR | | |
| 295 | 1 | | | NR | | |
| 298 | 2 | | | TEHKNR | Q | |
| 301 | 1 | S | | K | | |
| 302 | 3 | | D | SEKQT | L | |
| 303 | 3 | DT | | SAGQY | | |
| 304 | 2 | NQ | | G | | |
| 305 | 4 | E | | YDHKMNV | | AGQRST |
| 306 | 1 | A | | F | | |
| 310 | 4 | | IL | YCEGHKNQRST | D | |
| 312 | 3 | | | LIKMQRST | | |
| 313 | 2 | | T | NR | K | |
| 314 | 1 | | E | ND | | |
| 318 | 2 | | | KHQR | | |
| 319 | 2 | IMR | ST | D | | |
| 320 | 3 | G | DES | QKNR | H | |
| 321 | 2 | NRT | A | P | | |
| 322 | 2 | | R | SDQ | | |
| 323 | 2 | DE | AHQ | L | | |
| 326 | 1 | | | TS | | |
| 327 | 1 | M | | LI | | |
| 331 | 2 | CGTV | | HQ | | |
| 335 | 4 | | | PDGHILMNQT | K | |
| 336 | 2 | | | GAHK | | |
| 337 | 2 | PVY | | Q | | |
| 338 | 1 | | | S | A | |
| 339 | 2 | CFS | M | L | | T |
| 340 | 3 | | | QADGHKS | T | |
| 341 | 3 | | EHN | SDR | Q | |
| 342 | 2 | L | S | WQ | | |
| 345 | 1 | D | | P | | |
| 346 | 3 | AF | | W | | |
| 349 | 1 | QT | | P | | |
| 350 | 2 | | Q | LIMS | | |
| 353 | 2 | | | A | S | |
| 354 | 2 | G | T | FHLM | | |
| 355 | 2 | | | IT | | |
| 357 | 1 | | | TA | | |
| 358 | 1 | | | RH | | |
| 359 | 2 | | | QAD | | S |
| 360 | 2 | F | | EAQ | S | |
| 362 | 4 | | E | YAIS | DHKT | R |
| 364 | 3 | | M | CADH | ST | Q |
| 366 | 2 | D | | F | | |
| 367 | 2 | F | | YEHQ | | |
| 371 | 3 | | | YAHNT | R | |
| 374 | 2 | | | PAKQR | | |
| 376 | 2 | ADS | | Y | | |
| 377 | 2 | | | NKR | H | |
| 378 | 2 | AHMNS | | I | | |
| 379 | 3 | | | PHKRV | | |
| 380 | 3 | | | GDENPQR | K | |
| 381 | 2 | | S | LAIMQ | | |
| 382 | 1 | | R | K | | |
| 383 | 3 | | | SIRT | L | |
| 384 | 1 | Y | | K | | |
| 385 | 2 | | | ILM | | |
| 386 | 2 | | | DQ | | |
| 387 | 2 | T | | PAR | | |
| 391 | 2 | | | A | S | |
| 393 | 2 | | | RHK | T | |
| 394 | 2 | | | DAELQ | ST | |
| 395 | 2 | | | YFHNQ | R | |
| 396 | 2 | | | A | GS | |
| 399 | 3 | | | TEIKPQRS | | |
| 400 | 2 | | | QEHN | | |
| 401 | 4 | | I | RDEHKLNQST | A | |
| 404 | 1 | | | IES | | |
| 405 | 1 | S | | D | | |
| 406 | 1 | | | HD | | |
| 407 | 2 | | K | QAFR | | |
| 410 | 2 | | | IA | | |
| 413 | 2 | | | TS | | |
| 415 | 1 | | | E | A | |
| 417 | 4 | | | IDEGKLMQSTVY | D | |
| 418 | 4 | | | DHQS | | |
| 419 | 3 | | | TACGHKNQRS | AG | |
| 420 | 3 | | | KFHLNQRSY | | D |
| 421 | 4 | | | PACDEFGHIKLNTWY | A | |
| 422 | 4 | | | NACDHILQRSTWY | | |
| 423 | 1 | | | SAD | | |
| 424 | 1 | | | GS | | |
| 425 | 3 | | | LAFITVY | | |
| 427 | 2 | | | AGT | | |
| 429 | 1 | | | IET | | |
| 430 | 1 | D | | T | | |
| 433 | 4 | DE | KSV | PT | AQ | |
| 434 | 3 | HIKNTV | AQ | G | | |
| 435 | 2 | CMT | | G | | |
| 436 | 2 | | | SDKLNQT | | |
| 438 | 4 | | | WCFGHIKLMNPQRSTY | | |
| 440 | 4 | | R | YCEFHLPQSTW | DGN | |
| 442 | 1 | | | GD | | |
| 443 | 4 | | | KACDEFGHQRSTWY | | |
| 444 | 4 | | | KCDFGILMNPRSTVWY | | |
| 447 | 2 | | | GDHNQT | | |
| 448 | 4 | | | KACDGNQRST | | |
| 449 | 4 | | | VACDEGHKMPTY | Q | |
| 451 | 4 | | | YDEGKLNSTV | ACQR | |
| 453 | 4 | | | LACDEFGHINSTVWY | KQR | |
| 454 | 1 | | | TAQ | | |
| 455 | 2 | | | GADKNS | | |
| 456 | 1 | | H | NT | | |
| 457 | 2 | | QTW | RAL | | |
| 458 | 3 | | | SADFKQTVW | L | |
| 459 | 2 | C | | DEGHNQ | | |
| 460 | 2 | | | TAEILPQ | | |
| 461 | 2 | | | VAHQT | | |
| 462 | 2 | | | TDKPRV | | |
| 463 | 3 | | S | IAMV | | |
| 464 | 4 | | | NDGHKLMT | A | |
| 465 | 3 | | I | AGNPQSTWY | | |
| 466 | 2 | | | DEQST | | |
| 468 | 4 | | | WACFGHLMNQRTY | | |
| 469 | 1 | | | GAS | | |
| 470 | 3 | | C | EADHIQST | | |
| 471 | 2 | | | FMY | | |
| 472 | 2 | | D | KAIRT | | |
| 474 | 3 | | | NADEFIKMS | LT | |
| 475 | 4 | EKR | | GDN | S | AQ |
| 476 | 4 | ANPQSTVY | | G | | |
| 477 | 2 | HKR | | S | | |
| 478 | 4 | A | I | VCLMW | NQST | |
| 479 | 2 | C | | SAGT | | |
| 482 | 3 | | | VL | H | |
| 483 | 3 | | KR | ACFGQSW | D | |
| 484 | 2 | | | KADGHNQS | | |

TABLE 4.4
Productivity score for selected positions in the Amy TS23t protein.

| | Productivity | VARIANTS SUITABILITY SCORE | | | | |
|---|---|---|---|---|---|---|
| POS | Score | (+) | (++) | (+++) WT AA 1^{ST} | (++++) | (+++++) |
| 12 | 2 | | S | YDHQT | G | |
| 13 | 3 | | S | FY | | |
| 20 | 3 | | | DN | | |
| 23 | 4 | | | LAEFGMNSTWY | Q | |
| 27 | 3 | | | VEGNST | Q | |
| 31 | 3 | | | AGHNPTY | QS | |
| 33 | 2 | | | NDGP | | |
| 34 | 2 | | | LHNQ | | |
| 40 | 1 | | | TR | | |
| 41 | 2 | | | ADQRS | | |
| 43 | 1 | | | WY | | |
| 44 | 1 | | | LT | | |
| 48 | 1 | | | YQT | | |
| 63 | 2 | | S | LV | EM | |
| 64 | 1 | | | YT | S | |
| 66 | 1 | | | L | I | |
| 68 | 1 | N | | ED | | |
| 74 | 1 | LW | | T | | |
| 79 | 1 | | | YHW | | |
| 81 | 1 | | | TH | | |
| 88 | 1 | | | AS | | |
| 89 | 2 | | A | IMV | | |
| 92 | 1 | | | AG | | |
| 96 | 2 | | K | GACE | | |
| 97 | 2 | | | MFV | | |
| 106 | 3 | D | | N | | |
| 109 | 1 | ET | | A | | |
| 117 | 2 | W | AS | VEQ | P | |
| 120 | 2 | AN | | V | | |
| 122 | 1 | | | V | | C |
| 133 | 2 | | H | GN | DP | E |
| 139 | 1 | | | AP | | |
| 146 | 1 | | | PA | | |
| 173 | 1 | | | LQW | | |
| 180 | 4 | CG | W | RIMW | | ADKNQT |
| 181 | 3 | CDHMPQT | | S | | AG |
| 194 | 2 | | | NF | H | |
| 209 | 4 | MNQR | | HEY | A | CW |
| 212 | 1 | P | | V | | |
| 213 | 1 | | | VT | | P |
| 215 | 1 | | H | E | | |
| 218 | 1 | | | NS | | |
| 223 | 2 | L | S | YF | | |
| 224 | 2 | | | VKR | Q | |
| 225 | 1 | | | ND | | |
| 226 | 3 | | | TAHNQ | | |
| 229 | 2 | | S | IV | | |
| 232 | 1 | | | FW | | |
| 242 | 1 | F | | Y | | |
| 243 | 2 | A | | S | | DQ |
| 244 | 3 | | | FHV | NS | LMT |
| 245 | 4 | | T | FNSV | | AMQ |
| 249 | 1 | | | LQ | | |
| 251 | 2 | P | | YHMQRS | | |
| 252 | 1 | | | VL | | |
| 253 | 2 | | H | RKT | | |
| 256 | 2 | | DQRY | T | N | |
| 258 | 2 | C | N | KQ | | |
| 260 | 1 | | | LM | R | |
| 262 | 2 | M | | AG | | |
| 271 | 4 | ADGW | HQ | VY | IPRS | MT |
| 282 | 2 | LM | E | N | | D |
| 285 | 2 | | A | M | QTV | I |
| 286 | 2 | | | SDHQT | NR | |
| 287 | 1 | | | LI | | |
| 294 | 2 | E | | NR | | |
| 295 | 1 | | | NR | | |
| 302 | 3 | | D | SEKQT | L | |
| 304 | 2 | NQ | | G | | |
| 312 | 3 | | | LIKMQRST | | |
| 313 | 2 | | T | NR | K | |
| 321 | 2 | NRT | A | P | | |
| 326 | 1 | | | TS | | |
| 327 | 1 | M | | LI | | |
| 331 | 2 | CGTV | | HQ | | |
| 335 | 4 | | | PDGHILMNQT | K | |
| 341 | 3 | | EHN | SDR | Q | |
| 342 | 2 | L | S | WQ | | |
| 345 | 1 | D | | P | | |
| 346 | 3 | AF | | W | | |
| 349 | 1 | QT | | P | | |
| 350 | 2 | | Q | LIMS | | |
| 353 | 2 | | | A | S | |
| 354 | 2 | G | T | FHLM | | |
| 355 | 2 | | | IT | | |
| 357 | 1 | | | TA | | |
| 358 | 1 | | | RH | | |
| 360 | 2 | F | | EAQ | S | |
| 362 | 4 | | E | YAIS | DHKT | R |
| 364 | 3 | | M | CADH | ST | Q |
| 366 | 2 | D | | F | | |
| 367 | 2 | F | | YEHQ | | |
| 371 | 3 | | | YAHNT | R | |
| 378 | 2 | AHMNS | | I | | |
| 380 | 3 | | | GDENPQR | K | |
| 383 | 3 | | | SIRT | L | |
| 384 | 1 | Y | | K | | |
| 385 | 2 | | | ILM | | |
| 386 | 2 | | | DQ | | |
| 387 | 2 | T | | PAR | | |
| 391 | 2 | | | A | S | |
| 395 | 2 | | | YFHNQ | R | |
| 396 | 2 | | | A | GS | |
| 400 | 2 | | | QEHN | | |
| 404 | 1 | | | IES | | |
| 405 | 1 | S | | D | | |
| 406 | 1 | | | HD | | |
| 410 | 2 | | | IA | | |
| 413 | 2 | | | TS | A | |
| 415 | 1 | | | E | D | |
| 417 | 4 | | | IDEGKLMQSTVY | | |
| 418 | 4 | | | DHQS | AG | |
| 420 | 3 | | | KFHLNQRSY | A | |
| 421 | 4 | | | PACDEFGHIKLNTWY | | |
| 422 | 4 | | | NACDHILQRSTWY | | |
| 423 | 1 | | | SAD | | |
| 424 | 1 | | | GS | | |
| 425 | 3 | | | LAFITVY | | |
| 427 | 2 | | | AGT | | |
| 429 | 1 | | | IET | | |
| 430 | 1 | D | | T | | |
| 434 | 3 | HIKNTV | AQ | G | | |
| 435 | 2 | CMT | | G | | |
| 440 | 4 | | R | YCEFHLPQSTW | DGN | |
| 442 | 1 | | | GD | | |
| 443 | 4 | | | KACDEFGHQRSTWY | | |
| 449 | 4 | | | VACDEGHKMPTY | Q | |
| 454 | 1 | | | TAQ | | |
| 455 | 2 | | | GADKNS | | |
| 456 | 1 | | H | NT | | |
| 457 | 2 | | QTW | RAL | | |
| 458 | 3 | | | SADFKQTVW | L | |
| 460 | 2 | | | TAEILPQ | | |
| 461 | 2 | | | VAHQT | | |
| 462 | 2 | | | TDKPRV | | |
| 463 | 3 | | S | IAMV | | |
| 464 | 4 | | | NDGHKLMT | A | |
| 466 | 2 | | | DEQST | | |
| 468 | 4 | | | WACFGHLMNQRTY | | |
| 469 | 1 | | | GAS | | |
| 471 | 2 | | | FMY | | |
| 472 | 2 | | D | KAIRT | | |
| 474 | 3 | | | NADEFIKMS | LT | |
| 476 | 4 | ANPQSTVY | | G | | |
| 477 | 2 | HKR | | S | | |
| 478 | 4 | A | I | VCLMW | NQST | |
| 479 | 2 | C | | SAGT | | |
| 482 | 3 | | | VL | H | |

TABLE 4.5
Productivity score for selected positions in the
Amy TS23t protein.

| | Productivity | VARIANTS SUITABILITY SCORE | | | | |
|---|---|---|---|---|---|---|
| POS | score | (+) | (++) | (+++) WT AA 1^{ST} | (++++) | (+++++) |
| 1 | 2 | | | NHK | QR | |
| 2 | 2 | | A | TEQ | | |
| 3 | 2 | W | G | ADEHPT | | |
| 5 | 4 | | | IDEHKNRTVY | S | |
| 7 | 2 | R | N | EHKP | Q | |
| 15 | 2 | | AH | WY | | |
| 16 | 1 | | | DE | | |
| 17 | 4 | | | LCDEFGHIMNQRSTV | AY | |
| 18 | 2 | E | | PQ | H | |
| 19 | 3 | G | | NDEHPS | AKT | R |
| 22 | 3 | C | DL | TAHQ | KS | |
| 25 | 3 | | | TDGLNPSW | H | |
| 26 | 2 | | | KAGHRS | N | |
| 28 | 3 | | D | KGHNQRST | | |
| 29 | 3 | | | NDGKPRST | AH | |
| 30 | 3 | | | ED | | |
| 32 | 1 | | | AGP | | |
| 36 | 2 | | D | SPQT | | |
| 37 | 3 | | | LHIMQRSTVY | | |
| 50 | 2 | D | | GS | A | |
| 51 | 1 | | R | T | Q | |
| 52 | 1 | | KR | S | | |
| 53 | 2 | | K | QAS | | |
| 54 | 3 | | W | SDEHKNRT | AQ | |
| 56 | 4 | | | VAEHINPQRST | | |
| 59 | 2 | CK | | GS | | |
| 60 | 2 | | IL | VQ | GT | AS |
| 70 | 3 | | | NAHKPQRS | D | |
| 71 | 1 | | | QAS | | |
| 73 | 2 | | AH | GQR | D | |
| 75 | 4 | | LT | IAEGHKMRSW | FQ | |
| 82 | 2 | | I | KAEFLNY | | |
| 83 | 3 | | | TADFHIKLQWY | | |
| 87 | 2 | | | QLNSTW | P | |
| 90 | 2 | | | QAGH | | |
| 91 | 3 | | E | AFGHIRW | K | |
| 93 | 1 | | R | K | | |
| 94 | 3 | | | AFHILSTW | R | |
| 95 | 3 | | | AGLQRST | IK | |
| 103 | 1 | | I | VA | | |
| 105 | 1 | I | | F | | |
| 107 | 1 | CS | | H | | |
| 108 | 2 | | AL | KR | | |
| 110 | 1 | | | GAS | | |
| 113 | 3 | | LT | GER | AHQ | D |
| 114 | 1 | R | | TS | | |
| 116 | 2 | W | N | FAHIQR | | |
| 118 | 2 | AKS | | D | | |
| 123 | 1 | | | DAS | | |
| 125 | 2 | | K | SQ | FHR | |
| 126 | 1 | | | N | D | |
| 128 | 2 | C | Y | N | W | |
| 129 | 3 | | LMT | QAV | KR | I |
| 131 | 1 | | | T | | I |
| 134 | 2 | | DS | TA | P | |
| 135 | 2 | HIKLM | | Y | | |
| 136 | 2 | CHT | AGPR | Q | | |
| 138 | 2 | CLMT | DN | Q | | |
| 140 | 1 | | | WHK | | |
| 142 | 3 | | GHTV | KAILQSW | | |
| 144 | 1 | H | | D | | |
| 147 | 2 | | AL | GH | | W |
| 149 | 3 | | | GAHILQRWY | D | |
| 150 | 2 | | GK | N | D | |
| 152 | 1 | S | | YH | | |
| 154 | 2 | FIV | K | SNQ | G | |
| 156 | 1 | | F | KR | | |
| 158 | 2 | | A | RKW | LS | |
| 160 | 4 | | M | YS | AEKLPQRTVW | DIN |
| 162 | 1 | M | | F | | |
| 165 | 2 | DF | C | T | | IV |
| 166 | 3 | CGHPQ | | D | | |
| 167 | 2 | ANS | L | W | | |
| 168 | 2 | N | | D | | |
| 169 | 2 | | | EFHIQT | A | |
| 170 | 4 | FL | CDM | SAQTWY | H | |
| 171 | 1 | | L | R | | |
| 172 | 3 | | | KADLNQRY | F | |
| 174 | 4 | | | NACDS | HQT | R |
| 175 | 2 | CL | K | R | | |
| 177 | 2 | H | | YFW | | |
| 178 | 2 | AI | | KG | | LQ |
| 179 | 1 | I | | F | | |
| 182 | 4 | H | VY | TW | FR | ACDEGLMNPQ |
| 183 | 1 | EN | | G | | |
| 185 | 3 | C | G | ANS | Q | DE |
| 192 | 1 | I | D | T | | |
| 195 | 2 | M | V | GA | H | C |
| 197 | 4 | AN | F | Y | | D |
| 202 | 2 | | GH | FLMNY | S | |
| 203 | 2 | CKN | E | AD | Q | |
| 207 | 1 | | | M | Y | |
| 210 | 4 | | LM | P | CEGHKNQT | DR |
| 214 | 3 | | | TAKQRS | DE | |
| 217 | 2 | AS | Q | K | | |
| 221 | 4 | | EW | THNP | ADKQRSY | |
| 228 | 3 | | | NDEFGHL | PQS | |
| 234 | 2 | | GS | LIM | | A |
| 236 | 1 | E | | A | | |
| 237 | 1 | | I | V | | |
| 246 | 3 | | F | PK | DQRS | AEY |
| 250 | 4 | | I | TADEGHKQRY | | |
| 254 | 3 | | AEH | NDKMQRT | | |
| 255 | 1 | | | QR | | |
| 257 | 2 | | | GDKNRST | | |
| 264 | 1 | | | GS | | |
| 268 | 2 | | | SN | T | |
| 269 | 2 | EHLM | AS | Y | | |
| 272 | 3 | | HQ | NEMR | ADKST | |
| 273 | 1 | R | | K | | |
| 275 | 2 | D | R | HQ | | |
| 279 | 3 | | | TDHNQRS | | |
| 283 | 3 | DFT | Q | GE | AHKNRS | |
| 284 | 2 | | | SDEHKRT | Q | |
| 298 | 2 | | | TEHKNR | Q | |
| 301 | 1 | S | | K | | |
| 303 | 3 | DT | | SAGQY | | |
| 305 | 4 | E | | YDHKMNV | | AGQRST |
| 306 | 1 | A | | F | | |
| 310 | 4 | | IL | YCEGHKNQRST | D | |
| 314 | 1 | | E | ND | | |
| 318 | 2 | | | KHQR | | |
| 319 | 2 | IMR | ST | D | | |
| 320 | 3 | G | DES | QKNR | H | |
| 322 | 2 | | R | SDQ | | |
| 323 | 2 | DE | AHQ | L | | |
| 336 | 2 | | | GAHK | | |
| 337 | 2 | PVY | | Q | | |
| 338 | 1 | | | S | A | |
| 339 | 2 | CFS | M | L | | T |
| 340 | 3 | | | QADGHKS | T | |
| 359 | 2 | | | QAD | | S |
| 374 | 2 | | | PAKQR | | |
| 376 | 2 | ADS | | Y | | |
| 377 | 2 | | | NKR | H | |
| 379 | 3 | | | PHKRV | | |
| 381 | 2 | | S | LAIMQ | | |
| 382 | 1 | | R | K | | |
| 393 | 2 | | | RHK | T | |
| 394 | 2 | | | DAELQ | ST | |
| 399 | 3 | | | TEIKPQRS | | |
| 401 | 4 | | I | RDEHKLNQST | A | |
| 407 | 2 | | K | QAFR | | |
| 419 | 3 | | | TACGHKNQRS | | D |
| 433 | 4 | DE | KSV | PT | AQ | |
| 436 | 2 | | | SDKLNQT | | |
| 438 | 4 | | | WCFGHIKLMNPQRSTY | | |
| 444 | 4 | | | KCDFGILMNPRSTVWY | | |
| 447 | 2 | | | GDHNQT | | |
| 448 | 4 | | | KACDGNQRST | | |
| 451 | 4 | | | YDEGKLNSTV | ACQR | |
| 453 | 4 | | | LACDEFGHINSTVWY | KQR | |
| 459 | 2 | C | | DEGHNQ | | |
| 465 | 3 | | I | AGNPQSTWY | | |
| 470 | 3 | | C | EADHIQST | | |
| 475 | 4 | EKR | | GDN | S | AQ |
| 483 | 3 | | KR | ACFGQSW | D | |
| 484 | 2 | | | KADGHNQS | | |

All references cited herein are herein incorporated by reference in their entirety for all purposes.

1. A variant α-amylase polypeptide, comprising at least one combinable mutation at a productive amino acid position; wherein:
(i) the combinable mutation is a mutation that improves at least one desirable property of the variant α-amylase compared to the parental α-amylase, while not significantly decreasing either expression, activity, or stability of the variant α-amylase, compared to the parental α-amylase,
(ii) the productive position is an amino acid position that can be substituted with a plurality of different amino acid residues, all of which substitutions result in a variant α-amylase that meets the requirements of (i), and
(iii) the combinable mutation is listed in Table C or Table D, which uses SEQ ID NO: 2 for numbering.

2. The variant amylase of claim 1, wherein the combinable mutation has a performance property listed in Table A.

3. The variant amylase of claim 1, wherein the combinable mutation produces a variant wherein the minimum performance indices (PI) relative to the parental amylase for (i) protein expression, (ii) activity, (iii) microswatch activity, and (iv) detergent stability or thermostability are greater than or equal to 0.9, and in addition the PI for any one of these properties is greater than or equal to 1.0.

4. The variant amylase of claim 1, wherein the combinable mutation produces a variant wherein the minimum performance indices (PI) relative to the parental amylase for (i) protein expression, (ii) activity, (iii) microswatch activity, and (iv) detergent stability or thermostability are greater than or equal to 0.8, and in in addition have a PI for any one of these tests that is greater than or equal to 1.2.

5. The variant amylase of claim 1, wherein the combinable mutation produces a variant wherein the minimum performance indices (PI) relative to the parental amylase for (i) protein expression, (ii) activity, (iii) microswatch activity, and (iv) detergent stability or thermostability are greater than or equal to 0.5, and in in addition have a PI for any one of these tests that is greater than or equal to 1.5.

6. The variant amylase of claim 1, wherein the combinable mutation has a sustainability score of +++, ++++, or +++++.

7. The variant amylase of claim 1, wherein the combinable mutation has a sustainability score of ++++, or +++++.

8. The variant amylase of claim 1, wherein the combinable mutation has a sustainability score of +++++.

9. The variant amylase of claim 1, wherein the combinable mutation has a productivity score of 1 or 2.

10. The variant amylase of claim 1, wherein the parental α-amylase has at least 60% amino acid sequence identity to the amino acid sequence of SEQ ID NO: 1 or SEQ ID NO: 2.

11. The variant amylase of claim 1, wherein the parental α-amylase has at least 70% amino acid sequence identity to the amino acid sequence of SEQ ID NO: 1 or SEQ ID NO: 2.

12. The variant amylase of claim 1, wherein the parental α-amylase has at least 80% amino acid sequence identity to the amino acid sequence of SEQ ID NO: 1 or SEQ ID NO: 2.

13. The variant amylase of claim 1, wherein the parental α-amylase has at least 90% amino acid sequence identity to the amino acid sequence of SEQ ID NO: 1 or SEQ ID NO: 2.

14. A composition comprising the variant amylase of claim 1.

15. The composition of claim 14, wherein the composition is effective for removing starchy stains from laundry, dishes, or textiles.

16. The composition of claim 14, further comprising a surfactant.

17. The composition of claim 14, wherein the composition is a detergent composition.

18. The composition of claim 14, wherein the composition is a laundry detergent or a laundry detergent additive.

19. The composition of claim 14, wherein the composition is manual or automatic dishwashing detergent.

20. A method for removing a starchy stain or soil from a surface, comprising:
incubating the surface in the presence of a aqueous composition comprising an effective amount of the variant amylase of claim 1,
allowing the polypeptide to hydrolyze starch components present in the starchy stain to produce smaller starch-derived molecules that dissolve in the aqueous composition, and
rinsing the surface,
thereby removing the starchy stain from the surface.

21. The method of claim 20, wherein the aqueous composition further comprises a surfactant.

22. The method of claim 20, wherein the surface is a textile surface.

23. The method of claim 20, wherein the surface is on dishes.

24. The method of claim 20, wherein the surface is a soiled hard surface.

25. An isolated polynucleotide encoding a polypeptide of claim 1.

26. An expression vector comprising the polynucleotide of claim 25.

27. A host cell comprising the expression vector of claim 26.


SEQUENCE LISTING (2 sequences)

SEQ ID NO: 1 - protein, 583 aa, Bacillus sp.
  misc_feature: the mature form of Bacillus sp. strain TS-23 amylase
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAKTSNVTFTVNNATTTSGQNVYVVANIPELGNWNTANAIKMNPSSYPTWKATIALPQGKAIEFKFIKKDQAGNVIWESTSNRTYTVPFSSTGSYTASWNVP

SEQ ID NO: 2 - protein, 484 aa, Bacillus sp.
  misc_feature: mature form of a C-terminal truncated form of Bacillus sp. strain TS-23amylase
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK